(19) United States
(12) Patent Application Publication
(10) Pub. No.: US 2014/0314740 A1
(43) Pub. Date: Oct. 23, 2014

(54) COMPOSITIONS AND METHODS FOR THE PREVENTION OR TREATMENT OF DIABETIC COMPLICATIONS

(71) Applicant: The Johns Hopkins University, Baltimore, MD (US)

(72) Inventors: Peter A. Campochiaro, Baltimore, MD (US); Lili Lu, Baltimore, MD (US)

(73) Assignee: THE JOHNS HOPKINS UNIVERSITY, Baltimore, MD (US)

(21) Appl. No.: 14/363,358

(22) PCT Filed: Dec. 10, 2012
(86) PCT No.: PCT/US2012/068792

§ 371 Date: Jun. 6, 2014

Related U.S. Application Data
(60) Provisional application No. 61/568,735, filed on Dec. 9, 2011.

(51) Int. Cl. C07K 16/18; G01N 33/72; C12N 15/113; C12Q 1/54; A61K 31/713; A61K 31/352

(52) U.S. Cl. CPC C07K 16/18; A61K 31/713; A61K 31/352; C12N 15/1138; C12Q 1/54; G01N 33/723
USPC 424/130.1; 514/44 A; 514/455; 514/456; 435/320.1; 435/14; 435/29

(57) ABSTRACT

The invention provides compositions and methods for the prevention, amelioration, and/or treatment of diabetes complications, including ocular diseases associated with increased glucose transport and/or oxidative stress.

### CROSS-REFERENCE TO RELATED APPLICATIONS

This application claims the benefit of the following U.S. Provisional Application No. 61/568,735, filed Dec. 9, 2011, the entire contents of each of which are incorporated herein by reference.

### BACKGROUND

Diabetes is a prevalent disease that is becoming more prevalent over time. The major complications of diabetes are retinopathy, nephropathy, neuropathy, and acceleration of atherosclerosis. Hyperglycemia has been demonstrated to be the primary insult that leads to diabetic complications. In particular, tight control of serum glucose slows the progression of diabetic retinopathy. While the mechanism by which hyperglycemia leads to various complications is uncertain, it appears that compared to complication-resistant tissues, more glucose from blood is able to gain entry into susceptible tissues. Accordingly, methods for reducing glucose levels in susceptible tissues are urgently required to prevent or ameliorate the complications of diabetes.

### SUMMARY OF THE INVENTION

The invention provides compositions and methods for the prevention, amelioration, and/or treatment of diabetic complications, including retinopathy, nephropathy, neuropathy, or acceleration of atherosclerosis associated with increased glucose transport and/or oxidative stress. The invention further provides for the use of agents of the invention for the preparation of medicaments for the prevention, amelioration, and/or treatment of diabetic complications.

In one aspect, the invention provides a method of treating or preventing a diabetic complication in a subject (e.g., human patient), the method involving administering to a subject in need thereof an effective amount of a Glut1 antagonist that is any one or more of inhibitory nucleic acids, antibodies that specifically bind Glut1, and small compounds. In one embodiment, the diabetic complication is retinopathy, nephropathy, neuropathy, or acceleration of atherosclerosis.

In another aspect, the invention provides a method of treating or preventing diabetic retinopathy in a subject, the method involving administering to a subject in need thereof an effective amount of a Glut1 antagonist that is any one or more of inhibitory nucleic acids, antibodies that specifically bind GLUT1, and small compounds. In one embodiment, the inhibitory nucleic acid is: Homo sapiens siRNA 777 CCTTTTCGTTAACCGCTTT 795. In one embodiment, the small compound is myricetin, genistein, 1,9-dideoxyforskolin, and/or forskolin.

In another aspect, the invention provides an expression vector encoding a glut1 inhibitory nucleic acid molecule. In one embodiment, the vector encodes an inhibitory nucleic acid is a siRNA, shRNA, or antisense polynucleotide. In another embodiment, the vector is a adenoviral 2 or 9 vector.

In another aspect, the invention provides a method for monitoring Glut1 levels in a subject receiving a Glut1 antagonist, the method involving measuring glucose and/or glycohemoglobin levels in a red blood cells of the subject and comparing the levels to a reference. In one embodiment, the reference is the level of glucose and/or glycohemoglobin present in a healthy control subject. In another embodiment, the reference is the level of glucose and/or glycohemoglobin present in the subject prior to treatment with a Glut1 antagonist. In one embodiment, the reference is the level of glucose and/or glycohemoglobin present in the subject at an earlier time point. In another embodiment, a reduction in glucose and/or glycohemoglobin levels relative to the reference indicates that Glut1 antagonist therapy is efficacious. In another aspect, the invention provides an ocular formulation containing a Glut1 antagonist that is any one or a combination of inhibitory nucleic acids, antibodies that specifically bind Glut1, and small compounds. In one embodiment, the Glut1 antagonist is any one or more of inhibitory nucleic acids, antibodies that specifically bind Glut1, and small compounds. In another embodiment, the small compound is genistein, 1,9-dideoxyforskolin, and/or forskolin. In another embodiment, the inhibitory nucleic acid is a siRNA, shRNA, or antisense polynucleotide.

In various embodiments of the above aspects, the diabetic complication is retinopathy, nephropathy, neuropathy, or acceleration of atherosclerosis. In other embodiments, the subject is diagnosed as having or having a propensity to develop type I or type II diabetes, hyperglycemia, or a pre-diabetic condition. In particular embodiments of any aspect of the invention delineated herein the inhibitory nucleic acid is a siRNA, shRNA, or antisense polynucleotide. In particular embodiments, the inhibitory nucleic acid molecule is expressed in a vector. In still other embodiments, the Glut1 antagonist is administered orally or intra-ocularly. In still other embodiments, the Glut1 antagonist is administered systemically. In still other embodiments, the administration prevents elevation of superoxide radicals, increased expression of the chaperone protein β2 crystallin, and/or increased expression of vascular endothelial growth factor (VEGF).

### DEFINITIONS

“Active fragment” as in “active fragment of an enzyme” is understood as at least that portion of the enzyme that can catalyze the same reaction as the native, full length enzyme (e.g., inactivation of a peroxide, dismutation of superoxide into oxygen and hydrogen peroxide). In an embodiment, the active fragment of the enzyme has at least 50%, 60%, 70%, 80%, 90%, 100%, or more of the activity of the native full length enzyme. Activity can be determined by any of a number of enzyme kinetic parameters known to those of skill in the art, including, but not limited to, rate of product production by the active fragment as compared to the native, full length protein under the same conditions of substrate availability, temperature, etc. Methods to determine active fragments of enzymes is routine and well within the ability of those of skill in the art. Determination of active fragments can be performed initially using sequence alignments and other methods followed by routine enzyme kinetic experiments. Active fragments can include deletions of the amino acid sequence from the N-terminus or the C-terminus, or both. For example, an active fragment can have an N- and/or a C-terminal deletion of about 1, 2, 3, 4, 5, 6, 7, 8, 9, 10, 15, 20, 25, 30, 35, 40, 45, 50, 55, 60, 65, 70, 75, 80, 85, 90, 95, 100, or more amino acids. Active fragments can also include one or more internal deletions of the same exemplary lengths. Active fragments can also include one or more point mutations, particularly conservative point mutations, preferably outside of the catalytic center. At least an active fragment of an enzyme can include the full length, wild-type sequence of the enzyme.

As used herein, “active oxygen species” or “reactive oxygen species” are understood as understood as transfer of one or two electrons produces superoxide, an anion with the form O₂⁻, or peroxide anions, having the formula of O₂²⁻ or compounds containing an O—O single bond, for example hydrogen peroxides and lipid peroxides. Such superoxides and peroxides are highly reactive and can cause damage to cellular components including proteins, nucleic acids, and lipids.

An “agent” is understood herein to include a therapeutically active compound or a potentially therapeutic active compound, e.g., an antioxidant. An agent can be a previously known or unknown compound. As used herein, an agent is typically a non-cell based compound, however, an agent can include a biological therapeutic agent, e.g., peptide or nucleic acid therapeutic, e.g., siRNA, shRNA, cytokine, antibody, or other.

As used herein “amelioration” or “treatment” is understood as meaning to lessen or decrease at least one sign, symptom, indication, or effect of a specific disease or condition. For example, amelioration or treatment of diabetic retinopathy can be to reduce, delay, or eliminate one or more signs or symptoms of diabetic retinopathy including, but not limited to, a reduction in night vision, a reduction in overall visual acuity, a reduction in visual field, a reduction in the cone density in one or more quadrants of the retina, thinning of retina, particularly the outer nuclear layer, reduction in a- or b-wave amplitudes on scotopic or photopic electroretinograms (ERGs); or any other clinically acceptable indicators of disease state or progression. Amelioration and treatment can require the administration of more than one dose of an agent, either alone or in conjunction with other therapeutic agents and interventions. Amelioration or treatment does not require that the disease or condition be cured.

By “antagonist” is meant any agent that reduces the expression or activity of a polypeptide or polynucleotide that it targets.

“Antioxidant” as used herein is understood as a molecule capable of slowing or preventing the oxidation of other molecules. Oxidation is a chemical reaction that transfers electrons from a substance to an oxidizing agent. Such reactions can be promoted by or produce superoxide anions or peroxides. Oxidation reactions can produce free radicals, which start chain reactions that damage cells. Antioxidants terminate these chain reactions by removing free radical intermediates, and inhibit other oxidation reactions by being oxidized themselves. As a result, antioxidants are often reducing agents such as thiols, ascorbic acid or polyphenols. Antioxidants include, but are not limited to, α-tocopherol, ascorbic acid, Mn(III)tetrakis (4-benzoic acid) porphyrin, α-lipoic acid, and n-acetylcysteine.

As used herein, “changed as compared to a control” sample or subject is understood as having a level of the analyte or diagnostic or therapeutic indicator to be detected at a level that is statistically different than a sample from a normal, untreated, or control sample. Control samples include, for example, cells in culture, one or more laboratory test animals, or one or more human subjects. Methods to select and test control samples are within the ability of those in the art. An analyte can be a naturally occurring substance that is characteristically expressed or produced by the cell or organism (e.g., an active oxygen species, protein carbonyl content) or a substance produced by a reporter construct (e.g, β-galactosidase or luciferase). Depending on the method used for detection the amount and measurement of the change can vary. Changed as compared to a control reference sample can also include a change in night vision, overall visual acuity, size of visual field, cone density in the retina, thickness of the retina, particularly the outer nuclear layer of the retina, and reduction in a- or b-wave amplitudes on scotopic or ERGs. Determination of statistical significance is within the ability of those skilled in the art.

“Co-administration” as used herein is understood as administration of one or more agents to a subject such that the agents are present and active in the subject at the same time. Co-administration does not require a preparation of an admixture of the agents or simultaneous administration of the agents.

A “conservative amino acid substitution” is one in which the amino acid residue is replaced with an amino acid residue having a similar side chain. For example, families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Other conserved amino acid substitutions can also occur across amino acid side chain families, such as when substituting an asparagine for aspartic acid in order to modify the charge of a peptide. Thus, a predicted nonessential amino acid residue in a HR domain polypeptide, for example, is preferably replaced with another amino acid residue from the same side chain family or homologues across families (e.g. asparagine for aspartic acid, glutamine for glutamic acid). Conservative changes can further include substitution of chemically homologous non-natural amino acids (i.e. a synthetic non-natural hydrophobic amino acid in place of leucine, a synthetic non-natural aromatic amino acid in place of tryptophan).

“Contacting a cell” is understood herein as providing an agent to a test cell e.g., a cell to be treated in culture or in an animal, such that the agent or isolated cell can interact with the test cell or cell to be treated, potentially be taken up by the test cell or cell to be treated, and have an effect on the test cell or cell to be treated. The agent or isolated cell can be delivered to the cell directly (e.g., by addition of the agent to culture medium or by injection into the cell or tissue of interest), or by delivery to the organism by an enteral or parenteral route of administration for delivery to the cell by circulation, lymphatic, intraocular injection, intravitreal injection, subretinal injection, periocular injection or other means.

As used herein, “detecting”, “detection” and the like are understood that an assay performed for identification of a specific analyte in a sample, a product from a reporter construct or heterologous expression construct (e.g., viral vector) in a sample, or an activity of an agent in a sample. Detection can include the determination of oxidative damage in a cell or tissue, e.g., as determined by protein carbonyl content. Detection can include determination of cell density, particularly specific cell type cell density, cell viability/apoptosis, thickness of the retina, particularly the nuclear layer, photoreceptor function e.g, as determined by electroretinography, etc. The amount of analyte or activity detected in the sample can be none or below the level of detection of the assay or method.

By “diabetic complication” is meant any undesirable clinical change arising from or associated with diabetes, hyperglycemia, or a pre-diabetic condition.

By “diagnosing” as used herein refers to a clinical or other assessment of the condition of a subject based on observation, testing, or circumstances for identifying a subject having a disease, disorder, or condition based on the presence of at least one sign or symptom of the disease, disorder, or condition. Typically, diagnosing using the method of the invention includes the observation of the subject for other signs or symptoms of the disease, disorder, or condition.

The terms “effective amount,” or “effective dose” refers to that amount of an agent to produce the intended pharmacological, therapeutic or preventive result. The pharmacologically effective amount results in the amelioration of one or more signs or symptoms of a disease or condition or the advancement of a disease or condition, or causes the regression of the disease or condition. For example, a therapeutically effective amount preferably refers to the amount of a therapeutic agent that decreases the loss of night vision, the loss of overall visual acuity, the loss of visual field, by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or more as compared to an untreated control subject over a defined period of time, e.g., 2 weeks, one month, 2 months, 3 months, 6 months, one year, 2 years, 5 years, or longer. More than one dose may be required to provide an effective dose.

As used herein, the terms “effective” and “effectiveness” includes both pharmacological effectiveness and physiological safety. Pharmacological effectiveness refers to the ability of the treatment to result in a desired biological effect in the patient. Physiological safety refers to the level of toxicity, or other adverse physiological effects at the cellular, organ and/or organism level (often referred to as side-effects) resulting from administration of the treatment. On the other hand, the term “ineffective” indicates that a treatment does not provide sufficient pharmacological effect to be therapeutically useful, even in the absence of deleterious effects, at least in the unstratified population. (Such a treatment may be ineffective in a subgroup that can be identified by the expression profile or profiles.) “Less effective” means that the treatment results in a therapeutically significant lower level of pharmacological effectiveness and/or a therapeutically greater level of adverse physiological effects, e.g., greater liver toxicity.

Thus, in connection with the administration of a drug, a drug which is “effective against” a disease or condition indicates that administration in a clinically appropriate manner results in a beneficial effect for at least a statistically significant fraction of patients, such as a improvement of symptoms, a cure, a reduction in disease signs or symptoms, extension of life, improvement in quality of life, or other effect generally recognized as positive by medical doctors familiar with treating the particular type of disease or condition.

“Expression construct” as used herein is understood as a nucleic acid sequence including a sequence for expression as a polypeptide or nucleic acid (e.g., siRNA, shRNA) operably linked to a promoter and other essential regulatory sequences to allow for the expression of the polypeptide in at least one cell type. In a preferred embodiment, the promoter and other regulatory sequences are selected based on the cell type in which the expression construct is to be used. Selection of promoter and other regulatory sequences for protein expression are well known to those of skill in the art. An expression construction preferably also includes sequences to allow for the replication of the expression construct, e.g., plasmid sequences, virus sequences, etc. For example, expression constructs can be incorporated into replication competent or replication deficient viral vectors including, but not limited to, adenoviral (Ad) vectors, adeno-associated viral (AAV) vectors of all serotypes, self-complementary AAV vectors, and self-complementary AAV vectors with hybrid serotypes, self-complementary AAV vectors with hybrid serotypes and altered amino acid sequences in the capsid that provide enhanced transduction efficiency, lentiviral vectors, or plasmids for bacterial expression.

As used herein, “heterologous” as in “heterologous protein” is understood as a protein not natively expressed in the cell in which it is expressed, or a protein expressed from a nucleic acid that is not endogenous to the cell. For example, a heterologous protein is a protein expressed from a reporter construct, or a protein present in the cell that is expressed from an expression construct introduced into the cell, e.g. viral vector expression construct.

As used herein, the terms “identity” or “percent identity”, refers to the subunit sequence similarity between two polymeric molecules, e.g., two polynucleotides or two polypeptides. When a subunit position in both of the two molecules is occupied by the same monomeric subunit, e.g., if a position in each of two peptides is occupied by serine, then they are identical at that position. The identity between two sequences is a direct function of the number of matching or identical positions, e.g., if half (e.g., 5 positions in a polymer 10 subunits in length), of the positions in two peptide or compound sequences are identical, then the two sequences are 50% identical; if 90% of the positions, e.g., 9 of 10 are matched, the two sequences share 90% sequence identity. The identity between two sequences is a direct function of the number of matching or identical positions. Thus, if a portion of the reference sequence is deleted in a particular peptide, that deleted section is not counted for purposes of calculating sequence identity. Identity is often measured using sequence analysis software e.g., BLASTN or BLASTP (available at (www.ncbi.nih.gov/BLAST). The default parameters for comparing two sequences (e.g., “Blast”-ing two sequences against each other), by BLASTN (for nucleotide sequences) are reward for match=1, penalty for mismatch=−2, open gap=5, extension gap=2. When using BLASTP for protein sequences, the default parameters are reward for match=0, penalty for mismatch=0, open gap=11, and extension gap=1. Additional, computer programs for determining identity are known in the art.

As used herein, “isolated” or “purified” when used in reference to a polypeptide means that a naturally polypeptide or protein has been removed from its normal physiological environment (e.g., protein isolated from plasma or tissue) or is synthesized in a non-natural environment (e.g., artificially synthesized in an in vitro translation system or using chemical synthesis). Thus, an “isolated” or “purified” polypeptide can be in a cell-free solution or placed in a different cellular environment (e.g., expressed in a heterologous cell type). The term “purified” does not imply that the polypeptide is the only polypeptide present, but that it is essentially free (about 90-95%, up to 99-100% pure) of cellular or organismal material naturally associated with it, and thus is distinguished from naturally occurring polypeptide. Similarly, an isolated nucleic acid is removed from its normal physiological environment. “Isolated” when used in reference to a cell means the cell is in culture (i.e., not in an animal), either cell culture or organ culture, of a primary cell or cell line. Cells can be isolated from a normal animal, a transgenic animal, an animal having spontaneously occurring genetic changes, and/or an animal having a genetic and/or induced disease or condition. An isolated virus or viral vector is a virus that is removed from the cells, typically in culture, in which the virus was produced.

As used herein, “kits” are understood to contain at least one non-standard laboratory reagent for use in the methods of the invention. For example, a kit can include an expression construct for expression of a peroxidase and/or an active oxygen species metabolizing enzyme in the eye and instructions for use, all in appropriate packaging. The kit can further include any other components required to practice the method of the invention, as dry powders, concentrated solutions, or ready to use solutions. In some embodiments, the kit comprises one or more containers that contain reagents for use in the methods of the invention; such containers can be boxes, ampules, bottles, vials, tubes, bags, pouches, blister-packs, or other suitable container forms known in the art. Such containers can be made of plastic, glass, laminated paper, metal foil, or other materials suitable for holding reagents.

By “marker” is meant any analyte (e.g., polypeptide, polynucleotide) or other clinical parameter that is differentially present in a subject having a condition or disease as compared to a control subject (e.g., a person with a negative diagnosis or normal or healthy subject).

“Obtaining” is understood herein as manufacturing, purchasing, or otherwise coming into possession of.

As used herein, “operably linked” is understood as joined, preferably by a covalent linkage, e.g., joining an amino-terminus of one peptide, e.g., expressing an enzyme, to a carboxy terminus of another peptide, e.g., expressing a signal sequence to target the protein to a specific cellular compartment; joining a promoter sequence with a protein coding sequence, in a manner that the two or more components that are operably linked either retain their original activity, or gain an activity upon joining such that the activity of the operably linked portions can be assayed and have detectable activity, e.g., enzymatic activity, protein expression activity. Nucleic acid sequences can also be operably linked in tandem in an expression construct such that both polypeptide encoding sequences are transcribed from a single promoter sequence. Alternatively, each nucleic acid sequence encoding a polypeptide can be operably linked to a single promoter sequence.

“Oxidative stress related ocular disorders” as used herein include, but are not limited to, diabetic retinopathy, retinitis pigmentosa, macular degeneration including age related macular degeneration (AMD) both wet and dry, Lebers optic neuropathy, and optic neuritis.

The phrase “pharmaceutically acceptable carrier” is art recognized and includes a pharmaceutically acceptable material, composition or vehicle, suitable for administering compounds of the present invention to mammals. The carriers include liquid or solid filler, diluent, excipient, solvent or encapsulating material, involved in carrying or transporting the subject agent from one organ, or portion of the body, to another organ, or portion of the body. Each carrier must be “acceptable” in the sense of being compatible with the other ingredients of the formulation and not injurious to the patient. For example, pharmaceutically acceptable carriers for administration of cells typically is a carrier acceptable for delivery by injection, and do not include agents such as detergents or other compounds that could damage the cells to be delivered. Some examples of materials which can serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations, particularly phosphate buffered saline solutions which are preferred for intraocular delivery.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: water soluble antioxidants, such as ascorbic acid, cysteine hydrochloride, sodium bisulfate, sodium metabisulfite, sodium sulfite and the like; oil-soluble antioxidants, such as ascorbyl palmitate, butylated hydroxyanisole (BHA), butylated hydroxytoluene (BHT), lecithin, propyl gallate, α-tocopherol, and the like; and metal chelating agents, such as citric acid, ethylenediamine tetraacetic acid (EDTA), sorbitol, tartaric acid, phosphoric acid, and the like.

Formulations of the present invention include those suitable for oral, nasal, topical, transdermal, buccal, sublingual, intramuscular, intraperotineal, intraocular, intravitreal, subretinal, and/or other routes of parenteral administration. The specific route of administration will depend, inter alia, on the specific cell to be targeted. The formulations may conveniently be presented in unit dosage form and may be prepared by any methods well known in the art of pharmacy. The amount of active ingredient that can be combined with a carrier material to produce a single dosage form will generally be that amount of the compound that produces a therapeutic effect.

As used herein, “plurality” is understood to mean more than one. For example, a plurality refers to at least two, three, four, five, or more.

A “polypeptide” or “peptide” as used herein is understood as two or more independently selected natural or non-natural amino acids joined by a covalent bond (e.g., a peptide bond). A peptide can include 2, 3, 4, 5, 6, 7, 8, 9, 10, 11, 12, 13, 14, 15, 16, 17, 18, 19, 20, or more natural or non-natural amino acids joined by peptide bonds. Polypeptides as described herein include full length proteins (e.g., fully processed proteins) as well as shorter amino acids sequences (e.g., fragments of naturally occurring proteins or synthetic polypeptide fragments).

As used herein, “prevention” is understood as to limit, reduce the rate or degree of onset, or inhibit the development of at least one sign or symptom of a disease or condition particularly in a subject prone to developing the disease or disorder. For example, a subject having increased blood glucose is likely to develop diabetic retinopathy. The age of onset of one or more symptoms of the disease can sometimes be determined by the specific mutation. Prevention can include the delay of onset of one or more signs or symptoms of diabetic retinopathy and need not be prevention of appearance of at least one sign or symptom of the disease throughout the lifetime of the subject. Prevention can require the administration of more than one dose of an agent or therapeutic.

The diagnosis of retinitis pigmentosa relies upon documentation of progressive loss in photoreceptor function by electroretinography (ERG) and visual field testing. The mode of inheritance of RP is determined by family history. At least 35 different genes or loci are known to cause “nonsyndromic RP” (RP that is not the result of another disease or part of a wider syndrome). RP is commonly caused by a mutation in the opsin gene, but can be caused by mutations in a number of other genes expressed systemically or exclusively in the eye.

A “sample” as used herein refers to a biological material that is isolated from its environment (e.g., blood or tissue from an animal, cells, or conditioned media from tissue culture) and is suspected of containing, or known to contain an analyte, such as a virus, an antibody, or a product from a reporter construct. A sample can also be a partially purified fraction of a tissue or bodily fluid. A reference sample can be a “normal” sample, from a donor not having the disease or condition fluid, or from a normal tissue in a subject having the disease or condition (e.g., cells from a subject having a mutation that predisposes the subject to RP vs cells from a subject not having a mutation that predisposes the subject to RP). A reference sample can also be from an untreated donor or cell culture not treated with an active agent (e.g., no treatment or administration of vehicle only). A reference sample can also be taken at a “zero time point” prior to contacting the cell or subject with the agent or therapeutic intervention to be tested.

“Small molecule” as used herein is understood as a compound, typically an organic compound, having a molecular weight of no more than about 1500 Da, 1000 Da, 750 Da, or 500 Da. In an embodiment, a small molecule does not include a polypeptide or nucleic acid including only natural amino acids and/or nucleotides.

A “subject” as used herein refers to living organisms. In certain embodiments, the living organism is an animal. In certain preferred embodiments, the subject is a mammal. In certain embodiments, the subject is a domesticated mammal or a primate including a non-human primate. Examples of subjects include humans, monkeys, dogs, cats, mice, rats, cows, horses, goats, and sheep. A human subject may also be referred to as a patient.

A subject “suffering from or suspected of suffering from” a specific disease, condition, or syndrome has a sufficient number of risk factors or presents with a sufficient number or combination of signs or symptoms of the disease, condition, or syndrome such that a competent individual would diagnose or suspect that the subject was suffering from the disease, condition, or syndrome. Methods for identification of subjects suffering from or suspected of suffering from complications of diabetes are known in the art. Subjects suffering from, and suspected of suffering from, a specific disease, condition, or syndrome are not necessarily two distinct groups.

“Therapeutically effective amount,” as used herein refers to an amount of an agent which is effective, upon single or multiple dose administration to the cell or subject, in prolonging the survivability of the patient with such a disorder, reducing one or more signs or symptoms of the disorder, preventing or delaying and the like beyond that expected in the absence of such treatment.

An agent or other therapeutic intervention can be administered to a subject, either alone or in combination with one or more additional therapeutic agents or interventions, as a pharmaceutical composition in mixture with conventional excipient, e.g., pharmaceutically acceptable carrier, or therapeutic treatments.

The pharmaceutical agents may be conveniently administered in unit dosage form and may be prepared by any of the methods well known in the pharmaceutical arts, e.g., as described in Remington's Pharmaceutical Sciences (Mack Pub. Co., Easton, Pa., 1985). Formulations for parenteral administration may contain as common excipients such as sterile water or saline, polyalkylene glycols such as polyethylene glycol, oils of vegetable origin, hydrogenated naphthalenes and the like. In particular, biocompatible, biodegradable lactide polymer, lactide/glycolide copolymer, or polyoxyethylene-polyoxypropylene copolymers may be useful excipients to control the release of certain agents.

It will be appreciated that the actual preferred amounts of active compounds used in a given therapy will vary according to e.g., the specific compound being utilized, the particular composition formulated, the mode of administration and characteristics of the subject, e.g., the species, sex, weight, general health and age of the subject. Optimal administration rates for a given protocol of administration can be readily ascertained by those skilled in the art using conventional dosage determination tests conducted with regard to the foregoing guidelines.

As used herein, “susceptible to” or “prone to” or “predisposed to” or “having a propensity to develop” a specific disease or condition and the like refers to an individual who based on genetic, environmental, health, and/or other risk factors is more likely to develop a disease or condition than the general population. An increase in likelihood of developing a disease may be an increase of about 10%, 20%, 50%, 100%, 150%, 200%, or more.

Ranges provided herein are understood to be shorthand for all of the values within the range. This includes all individual sequences when a range of SEQ ID NOs: is provided. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

Unless specifically stated or obvious from context, as used herein, the term “or” is understood to be inclusive.

Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein can be modified by the term about.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

Other aspects of the invention are described in the following disclosure and are within the ambit of the invention.

### BRIEF DESCRIPTION OF THE FIGURES

The following Detailed Description, given by way of example, but not intended to limit the invention to specific embodiments described, may be understood in conjunction with the accompanying drawings.

FIGS. 1A-1G depict knockdown or blockade of GLUT1 reduced retinal glucose in diabetic mice. FIG. 1A is a Western Blot depicting GLUT1 expression in diabetic mice. Diabetic mice with sustained hyperglycemia for 3 weeks were given a 1 μl intraocular injection of vehicle or vehicle containing 1 μg of a pool of siRNA directed against SLC2A1 mRNA, which codes for GLUT1 on days 1, 4, and 7. On day 10 after injection, immunoblots showed strong knockdown of GLUT1 in retinas of siRNA-injected eyes compared to those injected with vehicle or those of nondiabetic mice. FIG. 1B shows that on day 10, the glucose concentration in retinal homogenates from siRNA-injected eyes (n=12) was significantly less (unpaired t-test) than that from vehicle-injected eyes. Bars represent the mean (±SEM). FIG. 1C shows treatment of diabetic mice for 2 weeks with 0.1-5.0 mg/kg of forskolin, an inhibitor of GLUT1, caused significant reduction in glucose levels in RBCs and retina. Bars represent the mean (±SEM) with n=10 for each. *P<0.05, for difference from retinas or RBC of forskolin-treated mice by ANOVA with Dunnett's correction for multiple comparisons. FIG. 1D shows that glycosylated hemoglobin in RBC from diabetics treated with 0.5 mg/kg of forskolin for 2 weeks was significantly less than that in untreated diabetics or nondiabetic mice. Bars represent the mean (±SEM) with n=4 for each with statistical comparison by unpaired t-test. FIG. 1E shows that one week after the onset of diabetes, mean blood glucose was high in forskolin-treated or untreated diabetics and was similarly high after 2 or 4 weeks of diabetes. Compared to non-diabetics, glucose levels were significantly elevated in RBC and retina after 1 week of diabetes with further elevation over time, but diabetics treated with 0.5 mg/kg/day of forskolin had no difference in retinal or RBC glucose levels compared to nondiabetics at 1, 2, or 4 weeks of diabetes. Each bar represents the mean (±SEM) with n=10 for each group. *P<0.05 for difference from RBCs of non-diabetic mice, **P<0.05 for difference from retinas of nondiabetic mice by ANOVA with Dunnett's correction for multiple comparisons. FIG. 1F shows that genistein, a tyrosine kinase inhibitor, and 1,9-dideoxyforskolin, which unlike forskolin does not stimulate adenylate cyclase, bind GLUT1. Diabetic mice given daily IP injections of 0.1 mg/kg of 1,9-dideoxyforskolin for 2 or 4 weeks or 50 mg/kg of genistein for 4 weeks had a significant reduction in retinal glucose level compared to untreated diabetics. Each bar represents the mean (±SEM) with n U 10 for each group. *P<0.05 for difference from drug-treated diabetics by ANOVA with Dunnett's correction for multiple comparisons. FIG. 1G is an immunoblot showing that compared to untreated diabetics, the level of GLUT1 protein in the retina was not reduced in diabetic mice treated with forskolin for 2 weeks.

FIGS. 2A-2D depict. Blockade of GLUT1 transport with forskolin suppresses markers of diabetic retinopathy. FIG. 2A shows that superoxide radicals were increased in the retinas of mice after 1 month of diabetes, and this was blocked by daily IP injections of 0.5 mg/kg of forskolin or 1,9-dideoxyforskolin. The level of superoxide radicals in retina was measured by fluorescence in retinal homogenates after administration of hydroethidine. Bars represent the mean (±SEM) with n=10 for each group. *P<0.05 for comparison with vehicle-treated diabetic mice by ANOVA with Dunnett's correction for multiple comparisons. FIG. 2B shows that the mRNA level for CRYBB2, which codes for the chaperone protein, β-crystallin, was significantly elevated in the retina after 1 month of diabetes, and this was blocked by daily IP injections of 0.5 mg/kg of forskolin. Bars represent the mean (±SEM) with n=10 for each group. Statistical comparison was made by unpaired t-test. FIG. 2C shows that CRYBB2 mRNA was also elevated in WBCs after 1 month of diabetes, and this was blocked by daily IP injections of 0.5 mg/kg of forskolin. Bars represent the mean (±SEM) with n=10 for each group. Statistical comparison was made by unpaired t-test. FIG. 2D shows an increase in retinal Vegf mRNA (measured by realtime quantitative RT-PCR) after 1 month of diabetes that was blocked by treatment with daily IP injections of 0.5 mg/kg of forskolin. Bars represent the mean (±SEM) with n=10 for each group. Statistical comparison was made by unpaired t-test.

FIGS. 3A-3D depict treatment with GLUT1 inhibitors decreased glucose levels in retina and RBCs 2 months after the onset of diabetes, and increased glucose levels in retina and RBCs without treatment with GLUT1 inhibitors. FIG. 3A shows treatment with GLUT1 inhibitors decreased glucose levels in retina and RBCs of diabetic mice given daily IP injections of 5 mg/kg of forskolin, 1 mg/kg of 1,9-dideoxyforskolin, compared to those without treatment. Mice were treated with streptozotocin and, after hyperglycemia was confirmed, they were randomized into groups that received daily IP injections of 5 mg/kg of forskolin, 1 mg/kg of 1,9-dideoxyforskolin, or no treatment. After 2 months of treatment and 5 hr after the last dose, control nondiabetic and diabetic mice were anesthetized, blood was aspirated from the heart, and retinas were dissected. Retina and RBC homogenates were assayed for protein content and glucose level. *P<0.05 for difference from RBC glucose in untreated diabetics; **P<0.05 for difference from retinal glucose in untreated diabetics by ANOVA with Dunnett's correction for multiple comparisons. ^{x}P<0.05 for difference from retinal glucose level in mice euthanized 5 hr after their last injection of drugs by ANOVA with Dunnett's correction for multiple comparisons. FIG. 3B shows treatment with GLUT1 inhibitors did not effect glucose levels in retina and RBCs of diabetic mice given daily IP injections of 5 mg/kg of forskolin, 1 mg/kg of 1,9-dideoxyforskolin, compared to those without treatment. Mice were treated with streptozotocin and, after hyperglycemia was confirmed, they were randomized into groups that received daily IP injections of 5 mg/kg of forskolin, 1 mg/kg of 1,9-dideoxyforskolin, or no treatment. After 2 months of treatment and 5 hr after the last dose, control nondiabetic and diabetic mice were anesthetized, and brains dissected. Brain homogenates were assayed for protein content and glucose level. FIG. 3C shows treatment with GLUT1 inhibitors decreased glucose levels in retina and RBCs of diabetic mice given a combination of 5 mg/kg forskolin, 50 mg/kg genistein, and 1 mg/kg myricetin, compared to those without treatment. Mice were treated with streptozotocin and, after hyperglycemia was confirmed, they were randomized into groups that were treated with a combination of 5 mg/kg forskolin, 50 mg/kg genistein, and 1 mg/kg myricetin, or received no treatment. After 2 months, protein and glucose levels were measured in retina and RBCs. Treated mice were divided into three groups that were euthanized 5, 24, or 48 hr after the last dose of drugs. Retina and RBC homogenates were assayed for protein content and glucose level. *P<0.05 for difference from RBC glucose in untreated diabetics; **P<0.05 for difference from retinal glucose in untreated diabetics by ANOVA with Dunnett's correction for multiple comparisons. ^{x}P<0.05 for difference from retinal glucose level in mice euthanized 5 hr after their last injection of drugs by ANOVA with Dunnett's correction for multiple comparisons. FIG. 3D shows treatment with GLUT1 inhibitors did not effect glucose levels in brain of diabetic mice given a combination of 5 mg/kg forskolin, 50 mg/kg genistein, and 1 mg/kg myricetin, compared to those without treatment. After 2 months, protein and glucose levels were measured in brain. Treated mice were divided into three groups that were euthanized 5, 24, or 48 hr after the last dose of drugs. Brain homogenates were assayed for protein content and glucose level.

FIG. 4 provides a comparison of mouse and human mRNA sequences.

FIG. 5 provides the sequence of mouse and human siRNAs.

### DETAILED DESCRIPTION

The invention generally features compositions and methods that are useful for the prevention or treatment of diabetic complications, including ocular diseases associated with increased glucose transport and/or oxidative stress (e.g., diabetic retinopathy).

High blood glucose results in high glucose levels in retina, because GLUT1, the sole glucose transporter between blood and retina, transports more glucose when blood glucose is high. This is the ultimate cause of diabetic retinopathy. As described in more detail below, the present invention is based, at least in part, on the discovery that knockdown of GLUT1 by intraocular injections of a pool of siRNAs directed against SLC2A1 mRNA which codes for GLUT1 significantly reduced mean retinal glucose levels in diabetic mice. Systemic treatment of diabetic mice with forskolin or genistein, which bind GLUT1 and inhibit glucose transport, significantly reduced retinal glucose to the same levels seen in non-diabetics. 1,9-Dideoxyforskolin, which binds GLUT1 but does not stimulate adenylate cyclase had an equivalent effect to that of forskolin regarding lowering retinal glucose in diabetics indicating that cyclic AMP is noncontributory.

GLUT1 inhibitors also reduced glucose and glycohemoglobin levels in red blood cells providing a peripheral biomarker for the effect. In contrast, brain glucose levels were not increased in diabetics and not reduced by forskolin. Treatment of diabetics with forskolin prevented early biomarkers of diabetic retinopathy, including elevation of superoxide radicals, increased expression of the chaperone protein b2 crystallin, and increased expression of vascular endothelial growth factor (VEGF). Accordingly, the invention provides methods for reducing Glut1 expression or activity as a promising therapeutic target for prevention of diabetic complications, including retinopathy.

### Diabetic Complications

Diabetic complications, including retinopathy, nephropathy, neuropathy, and acceleration of atherosclerosis arise in tissues that are susceptible to hyperglycemia. Glucose transport occurs through sodium-driven sugar cotransporters (SGLTs) or facilitative glucose transporters (GLUTs). Active transport by SGLTs is required for absorption of glucose in the gut or reabsorption in the kidney, while glucose movement within the body occurs primarily through 14 isoforms that constitute the GLUT protein family. A particularly prominent member of the family is GLUT1, which is ubiquitous and collaborates with other GLUTs for transport into most tissues, but is uniquely responsible for transport across the blood-retinal barrier (BRB). Another prominent member is GLUT4; its translocation to the plasma membrane in response to insulin is the rate-limiting step in glucose uptake into heart, skeletal muscle and fat.

Unlike GLUT4, which in the absence of insulin is located predominantly in the cytoplasm and is thus inactive, GLUT1 is localized predominantly at the plasma membrane. Although GLUT1 transport activity is regulated in ways other than its localization, compared to GLUT4, its transport activity is influenced to a greater degree by blood glucose concentration. The invention provides compositions and methods for reducing Glut1 transport activity. In particular, the invention provides inhibitory nucleic acids that bind a Glut1 or other Glut family member.

The sequence of an exemplary human GLUT1 protein (NCBI Accession NO. NP_{—}006507) follows:

| | |
|---|---|
| 1 | mepsskkltg rlmlavggav lgslqfgynt gvinapqkvi |
| | eefynqtwvh rygesilptt |
| 61 | lttlwslsva ifsvggmigs fsvglfvnrf grrnsmlmmn |
| | llafvsavlm gfsklgksfe |
| 121 | mlilgrfiig vycglttgfv pmyvgevspt alrgalgtlh |
| | qlgivvgili aqvfgldsim |
| 181 | gnkdlwplll siifipallq civlpfcpes prfllinrne |
| | enraksvlkk lrgtadvthd |
| 241 | lqemkeesrq mmrekkvtil elfrspayrq piliavvlql |
| | sqqlsginav fyystsifek |
| 301 | agvqqpvyat igsgivntaf tvvslfvver agrrtlhlig |
| | lagmagcail mtialalleq |
| 361 | lpwmsylsiv aifgfvaffe vgpgpipwfi vaelfsqgpr |
| | paaiavagfs nwtsnfivgm |
| 421 | cfqyveglcg pyvfiiftvl lvlffiftyf kvpetkgrtf |
| | deiasgfrqg gasqsdktpe |
| 481 | elfhplgads qv |

The sequence of an exemplary human glut1 polynucleotide (NCBI Ref: NM_{—}006516) follows:
Homo sapiens Solute Carrier Family 2 (Facilitated Glucose Transporter), Member 1 (SLC2A1), mRNA (3687 bp)

### NM_{—}006516.2

| | |
|---|---|
| 1 | tccaccattt tgctagagaa ggccgcggag gctcagagag gtgcgcacac ttgccctgag |
| 61 | tcacacagcg aatgccctcc gcggtcccaa cgcagagaga acgagccgat cggcagcctg |
| 121 | agcgaggcag tggttagggg gggccccggc cccggccact cccctcaccc cctccccgca |
| 181 | gagcgccgcc caggacaggc tgggccccag gccccgcccc gaggtcctgc ccacacaccc |
| 241 | ctgacacacc ggcgtcgcca gccaatggcc ggggtcctat aaacgctacg gtccgcgcgc |
| 301 | tctctggcaa gaggcaagag gtagcaacag cgagcgtgcc ggtcgctagt cgcgggtccc |
| 361 | cgagtgagca cgccagggag caggagacca aacgacgggg gtcggagtca gagtcgcagt |
| 421 | gggagtcccc ggaccggagc acgagcctga gcgggagagc gccgctcgca cgcccgtcgc |
| 481 | cacccgcgta cccggcgcag ccagagccac cagcgcagcg ctgccatgga gcccagcagc |
| 541 | 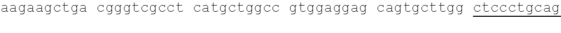 |
| 601 | 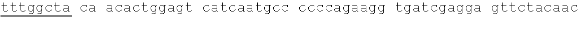 |
| 661 | cagacatggg tccaccgcta tggggagagc atcctgccca ccacgctcac cacgctctgg |
| 721 | 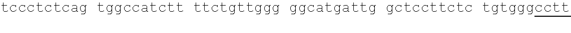 |
| 781 | 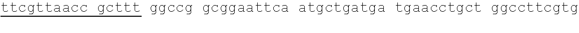 |
| 841 | tccgccgtgc tcatgggctt ctcgaaactg ggcaagtcct ttgagatgct gatcctgggc |
| 901 | cgcttcatca tcggtgtgta ctgcggcctg accacaggct tcgtgcccat gtatgtgggt |
| 961 | gaagtgtcac ccacagccct tcgtggggcc ctgggcaccc tgcaccagct gggcatcgtc |
| 1021 | gtcggcatcc tcatcgccca ggtgttcggc ctggactcca tcatgggcaa caaggacctg |
| 1081 | tggcccctgc tgctgagcat catcttcatc ccggccctgc tgcagtgcat cgtgctgccc |
| 1141 | ttctgccccg agagtccccg cttcctgctc atcaaccgca acgaggagaa ccgggccaag |
| 1201 | agtgtgctaa agaagctgcg cgggacagct gacgtgaccc atgacctgca ggagatgaag |
| 1261 | gaagagagtc ggcagatgat gcgggagaag aaggtcacca tcctggagct gttccgctcc |
| 1321 | cccgcctacc gccagcccat cctcatcgct gtggtgctgc agctgtccca gcagctgtct |
| 1381 | 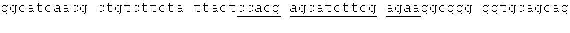 |
| 1441 | 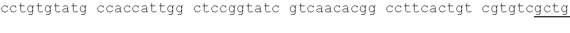 |
| 1501 | 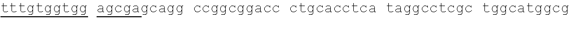 |
| 1561 | ggttgtgcca tactcatgac catcgcgcta gcactgctgg agcagctacc ctggatgtcc |
| 1621 | tatctgagca tcgtggccat ctttggcttt gtggccttct ttgaagtggg tcctggcccc |
| 1681 | atcccatggt tcatcgtggc tgaactcttc agccagggtc cacgtccagc tgccattgcc |
| 1741 | gttgcaggct tctccaactg gacctcaaat ttcattgtgg gcatgtgctt ccagtatgtg |
| 1801 | gagcaactgt gtggtcccta cgtcttcatc atcttcactg tgctcctggt tctgttcttc |
| 1861 | atcttcacct acttcaaagt tcctgagact aaaggccgga ccttcgatga gatcgcttcc |
| 1921 | ggcttccggc aggggggagc cagccaaagt gacaagacac ccgaggagct gttccatccc |
| 1981 | ctgggggctg attcccaagt gtgagtcgcc ccagatcacc agcccggcct gctcccagca |
| 2041 | gccctaagga tctctcagga gcacaggcag ctggatgaga cttccaaacc tgacagatgt |
| 2101 | cagccgagcc gggcctgggg ctcctttctc cagccagcaa tgatgtccag aagaatattc |
| 2161 | aggacttaac ggctccagga ttttaacaaa agcaagactg ttgctcaaat ctattcagac |
| 2221 | aagcaacagg ttttataatt tttttattac tgattttgtt atttttatat cagcctgagt |
| 2281 | ctcctgtgcc cacatcccag gcttcaccct gaatggttcc atgcctgagg gtggagacta |
| 2341 | agccctgtcg agacacttgc cttcttcacc cagctaatct gtagggctgg acctatgtcc |
| 2401 | taaggacaca ctaatcgaac tatgaactac aaagcttcta tcccaggagg tggctatggc |
| 2461 | cacccgttct gctggcctgg atctccccac tctaggggtc aggctccatt aggatttgcc |
| 2521 | ccttcccatc tcttcctacc caaccactca aattaatctt tctttacctg agaccagttg |
| 2581 | ggagcactgg agtgcaggga ggagagggga agggccagtc tgggctgccg ggttctagtc |
| 2641 | tcctttgcac tgagggccac actattacca tgagaagagg gcctgtggga gcctgcaaac |
| 2701 | tcactgctca agaagacatg gagactcctg ccctgttgtg tatagatgca agatatttat |
| 2761 | atatattttt ggttgtcaat attaaataca gacactaagt tatagtatat ctggacaagc |
| 2821 | caacttgtaa atacaccacc tcactcctgt tacttaccta aacagatata aatggctggt |
| 2881 | ttttagaaac atggttttga aatgcttgtg gattgagggt aggaggtttg gatgggagtg |
| 2941 | agacagaagt aagtggggtt gcaaccactg caacggctta gacttcgact caggatccag |
| 3001 | tcccttacac gtacctctca tcagtgtcct cttgctcaaa aatctgtttg atccctgtta |
| 3061 | cccagagaat atatacattc tttatcttga cattcaaggc atttctatca catatttgat |
| 3121 | agttggtgtt caaaaaaaca ctagttttgt gccagccgtg atgctcaggc ttgaaatgca |
| 3181 | ttattttgaa tgtgaagtaa atactgtacc tttattggac aggctcaaag aggttatgtg |
| 3241 | cctgaagtcg cacagtgaat aagctaaaac acctgctttt aacaatggta ccatacaacc |
| 3301 | actactccat taactccacc cacctcctgc acccctcccc acacacacaa aatgaaccac |
| 3361 | gttctttgta tgggcccaat gagctgtcaa gctgccctgt gttcatttca tttggaattg |
| 3421 | ccccctctgg ttcctctgta tactactgct tcatctctaa agacagctca tcctcctcct |
| 3481 | tcacccctga atttccagag cacttcatct gctccttcat cacaagtcca gttttctgcc |
| 3541 | actagtctga atttcatgag aagatgccga tttggttcct gtgggtcctc agcactattc |
| 3601 | agtacagtgc ttgatgcaca gcaggcactc agaaaatact ggaggaaata aaacaccaaa |
| 3661 | gatatttgtc aaaaaaaaaa aaaaaaa |

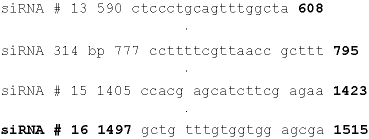

Mus musculus Solute Carrier Family 2 (Facilitated Glucose Transporter), Member 1 (Slc2a1), mRNA (2573 bp)

### NCBI Reference Sequence: NM_{—}011400.3

| | |
|---|---|
| 1 | tacaccccag aaccaatggc ggcggtccta taaaaaggca gctccgcgcg ctctccccca |
| 61 | agagcagagg cttgcttgta gagtgacgat ctgagctacg gggtcttaag tgcgtcaggg |
| 121 | cgtggaggtc tggcgggaga cgcatagtta cagcgcgtcc gttctccgtc tcgcagccgg |
| 181 | cacagctaga gcttcgagcg cagcgcggcc atggatccca gcagcaagaa ggtgacgggc |
| 241 | 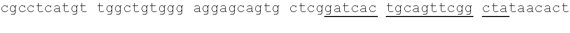 |
| 301 | ggtgtcatca acgcccccca gaaggttatt gaggagttct acaatcaaac atggaaccac |
| 361 | cgctacggag agcccatccc atccaccaca ctcaccacgc tttggtctct ctccgtggcc |
| 421 | 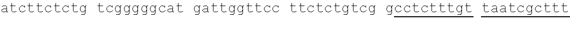 |
| 481 | ggcaggcgga actccatgct gatgatgaac ctgttggcct ttgtggctgc tgtgcttatg |
| 541 | ggcttctcca aactgggcaa gtcctttgag atgctgatcc tgggccgctt catcatcggt |
| 601 | gtgtactgcg gcctgactac tggctttgtg cccatgtatg tgggagaggt gtcacctaca |
| 661 | gctctacgtg gagccctagg cacactgcac cagctgggaa tcgtcgttgg catccttatt |
| 721 | gcccaggtgt ttggcttaga ctccatcatg ggcaatgcag acttgtggcc tctgctgctc |
| 781 | agtgtcatct tcatcccagc cctgctacag tgtatcctgt tgcccttctg ccccgagagc |
| 841 | ccccgcttcc tgctcatcaa tcgtaacgag gagaaccggg ccaagagtgt gctgaagaag |
| 901 | cttcgaggga cagccgatgt gacccgagac ctgcaggaga tgaaagaaga gggtcggcag |
| 961 | atgatgcggg agaagaaggt caccatcttg gagctgttcc gctcacccgc ctaccgccag |
| 1021 | cccatcctca tcgctgtggt gctgcagctg tcccagcagc tgtcgggtat caatgctgtg |
| 1081 | 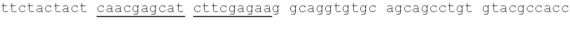 |
| 1141 | 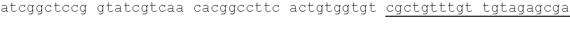 |
| 1201 | gctggacgac ggaccctgca cctcattggc ctggctggca tggcaggctg tgctgtgctc |
| 1261 | atgaccatcg ccctggcctt gctggaacgg ctgccttgga tgtcctatct gagcatcgtg |
| 1321 | gccatctttg gctttgtggc cttctttgaa gtaggccctg gtcctattcc atggttcatt |
| 1381 | gtggccgagc tgttcagcca ggggccccgt cctgctgcta ttgctgtggc tggcttctcc |
| 1441 | aactggacct caaacttcat tgtgggcatg tgcttccagt atgtggagca actgtgcggc |
| 1501 | ccctacgtct tcatcatctt cacggtgctc ctcgtgctct tcttcatctt cacctacttc |
| 1561 | aaagtccctg agaccaaagg ccgaaccttc gatgagatcg cttccggctt ccggcagggg |
| 1621 | ggtgccagcc aaagtgacaa gacacccgag gagctcttcc accctctggg ggcggactcc |
| 1681 | caagtgtgag gagccccaca cccagcccgg cctgctccct gcagcccaag gatctctctg |
| 1741 | gagcacaggc agctagatga gacctcttcc gaaccgacag atctcgggca agccgggcct |
| 1801 | gggcgccttt cctcagccag cagtgaagtc caggaggata ttcaggactt tgatggctcc |
| 1861 | agaattttta atgaaagcaa gactgctgct cagatctatt cagataagca gcaggtttta |
| 1921 | taattttttt attactgatt ttgttatttt ttttttttat cagccactct cctatctcca |
| 1981 | cactgtagtc ttcaccttga ttggcccagt gcctgagggt ggggaccacg ccctgtccag |
| 2041 | acacttgcct tctttgccaa gctaatctgt agggctggac ctatggccaa ggacacacta |
| 2101 | ataccgaact ctgagctagg aggctttacc gctggaggcg gtagctgcca cccacttccg |
| 2161 | caggcctgga cctcggcacc ataggggtcc ggactccatt ttaggattcg cccattcctg |
| 2221 | tctcttccta cccaaccact caattaatct ttccttgcct gagaccagtt ggaagcactg |
| 2281 | gagtgcaggg aggagaggga agggccaggc tgggctgcca ggttctagtc tcctgtgcac |
| 2341 | tgagggccac acaaacacca tgagaaggac ctcggaggct gagaacttaa ctgctgaaga |
| 2401 | cacggacact cctgccctgc tgtgtataga tggaagatat ttatatattt tttggttgtc |
| 2461 | aatattaaat acagacacta agttatagta tatctggaca aacccacttg taaatacacc |
| 2521 | aacaaactcc tgtaacttta cctaagcaga tataaatggc tggtttttag aaa |

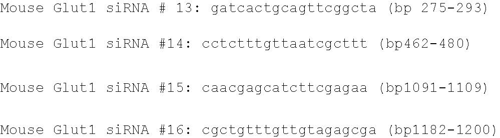

In one embodiment, a preferred murine siRNA is Mouse Glut1 siRNA #14: cctctttgttaatcgcttt (bp462-480)

### Inhibitory Nucleic Acid Molecules

Given that Glut1 is present in tissues that are susceptible to diabetes, the invention provides compositions that inhibit the expression or activity of Glut1, as well as methods of using such compositions for the prevention or treatment of diabetes complications. In one embodiment, the invention provides inhibitory nucleic acid molecules, such as antisense nucleic acid molecules, shRNAs, and siRNAs that decrease the expression of Glut1. Inhibitory nucleic acid molecules are essentially nucleobase oligomers that may be employed to decrease the expression of a target nucleic acid sequence, such as a nucleic acid sequence that encodes Glut1. The inhibitory nucleic acid molecules provided by the invention include any nucleic acid molecule sufficient to decrease the expression of a Glut1 nucleic acid molecule by at least 5-10%, desirably by at least 25%-50%, or even by as much as 75%-100%. Each of the nucleic acid sequences provided herein may be used, for example, in the discovery and development of therapeutic inhibitory nucleic acid molecules to decrease the expression of a Glut1 polynucleotide.

The invention is encompasses virtually any single-stranded or double-stranded nucleic acid molecule that decreases expression of a Glut1 polynucleotide. The invention further provides catalytic RNA molecules or ribozymes. Such catalytic RNA molecules can be used to inhibit expression of a Glut1 polynucleotide nucleic acid molecule in vivo.

The inclusion of ribozyme sequences within an antisense RNA confers RNA-cleaving activity upon the molecule, thereby increasing the activity of the constructs. The design and use of target RNA-specific ribozymes is described in Haseloff et al., Nature 334:585-591. 1988, and U.S. Patent Application Publication No. 2003/0003469 A1, each of which is incorporated by reference. In various embodiments of this invention, the catalytic nucleic acid molecule is formed in a hammerhead or hairpin motif. Examples of such hammerhead motifs are described by Rossi et al., Nucleic Acids Research and Human Retroviruses, 8:183, 1992. Example of hairpin motifs are described by Hampel et al., “RNA Catalyst for Cleaving Specific RNA Sequences,” filed Sep. 20, 1989, which is a continuation-in-part of U.S. Ser. No. 07/247,100 filed Sep. 20, 1988, Hampel and Tritz, Biochemistry, 28:4929, 1989, and Hampel et al., Nucleic Acids Research, 18: 299, 1990. These specific motifs are not limiting in the invention and those skilled in the art will recognize that all that is important in an enzymatic nucleic acid molecule of this invention is that it has a specific substrate binding site which is complementary to one or more of the target gene RNA regions, and that it have nucleotide sequences within or surrounding that substrate binding site which impart an RNA cleaving activity to the molecule.

In another approach, the inhibitory nucleic acid molecule is a double-stranded nucleic acid molecule used for RNA interference (RNAi)-mediated knock-down of the expression of a Glut1 polynucleotide. siRNAs are also useful for the inhibition of Glut1 polynucleotides. See, for example, Nakamoto et al., Hum Mol Genet, 2005. Desirably, the siRNA is designed such that it provides for the cleavage of a target Glut1 polynucleotide of the invention. In one embodiment, a double-stranded RNA (dsRNA) molecule is made that includes between eight and twenty-five (e.g., 8, 10, 12, 15, 16, 17, 18, 19, 20, 21, 22, 23, 24, 25) consecutive nucleobases of a nucleobase oligomer of the invention. The dsRNA can be two complementary strands of RNA that have duplexed, or a single RNA strand that has self-duplexed (small hairpin (sh)RNA). Typically, dsRNAs are about 21 or 22 base pairs, but may be shorter or longer (up to about 29 nucleobases) if desired. Double stranded RNA can be made using standard techniques (e.g., chemical synthesis or in vitro transcription). Kits are available, for example, from Ambion (Austin, Tex.) and Epicentre (Madison, Wis.). Methods for expressing dsRNA in mammalian cells are described in Brummelkamp et al. Science 296:550-553, 2002; Paddison et al. Genes & Devel. 16:948-958, 2002. Paul et al. Nature Biotechnol. 20:505-508, 2002; Sui et al. Proc. Natl. Acad. Sci. USA 99:5515-5520, 2002; Yu et al. Proc. Natl. Acad. Sci. USA 99:6047-6052, 2002; Miyagishi et al. Nature Biotechnol. 20:497-500, 2002; and Lee et al. Nature Biotechnol. 20:500-505 2002, each of which is hereby incorporated by reference. An inhibitory nucleic acid molecule that “corresponds” to a Glut1 polynucleotide comprises at least a fragment of the double-stranded gene, such that each strand of the double-stranded inhibitory nucleic acid molecule is capable of binding to the complementary strand of the target gene. The inhibitory nucleic acid molecule need not have perfect correspondence or need not be perfectly complementary to the reference sequence. In one embodiment, an siRNA has at least about 85%, 90%, 95%, 96%, 97%, 98%, or even 99% sequence identity with the target nucleic acid. For example, a 19 base pair duplex having 1-2 base pair mismatch is considered useful in the methods of the invention. In other embodiments, the nucleobase sequence of the inhibitory nucleic acid molecule exhibits 1, 2, 3, 4, 5 or more mismatches.

Inhibitory nucleic acid molecules of the invention also include double stranded nucleic acid “decoys.” Decoy molecules contain a binding site for a transcription factor that is responsible for the deregulated transcription of a gene of interest. The present invention provides decoys that competitively block binding to a regulatory element in a target gene (e.g., Glut1).

In one embodiment, the inhibitory nucleic acid molecules of the invention are administered systemically in dosages between about 1 and 100 mg/kg (e.g., 1, 5, 10, 20, 25, 50, 75, and 100 mg/kg). In other embodiments, the dosage ranges from between about 25 and 500 mg/m²/day. Desirably, a human patient having or at risk of developing a diabetic complication receives a dosage between about 50 and 300 mg/m²/day (e.g., 50, 75, 100, 125, 150, 175, 200, 250, 275, and 300).

### Modified Inhibitory Nucleic Acid Molecules

A desirable inhibitory nucleic acid molecule is one based on 2′-modified oligonucleotides containing oligodeoxynucleotide gaps with some or all internucleotide linkages modified to phosphorothioates for nuclease resistance. The presence of methylphosphonate modifications increases the affinity of the oligonucleotide for its target RNA and thus reduces the IC₅₀. This modification also increases the nuclease resistance of the modified oligonucleotide. It is understood that the methods and reagents of the present invention may be used in conjunction with any technologies that may be developed to enhance the stability or efficacy of an inhibitory nucleic acid molecule.

Inhibitory nucleic acid molecules include nucleobase oligomers containing modified backbones or non-natural internucleoside linkages. Oligomers having modified backbones include those that retain a phosphorus atom in the backbone and those that do not have a phosphorus atom in the backbone. For the purposes of this specification, modified oligonucleotides that do not have a phosphorus atom in their internucleoside backbone are also considered to be nucleobase oligomers. Nucleobase oligomers that have modified oligonucleotide backbones include, for example, phosphorothioates, chiral phosphorothioates, phosphorodithioates, phosphotriesters, aminoalkyl-phosphotriesters, methyl and other alkyl phosphonates including 3′-alkylene phosphonates and chiral phosphonates, phosphinates, phosphoramidates, thionophosphoramidates, thionoalkylphosphonates, thionoalkylphosphotriest-ers, and boranophosphates. Various salts, mixed salts and free acid forms are also included. Representative United States patents that teach the preparation of the above phosphorus-containing linkages include, but are not limited to, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; and 5,625,050, each of which is herein incorporated by reference.

Nucleobase oligomers having modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts. Representative United States patents that teach the preparation of the above oligonucleotides include, but are not limited to, U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439, each of which is herein incorporated by reference.

Nucleobase oligomers may also contain one or more substituted sugar moieties. Such modifications include 2′-O-methyl and 2′-methoxyethoxy modifications. Another desirable modification is 2′-dimethylaminooxyethoxy, 2′-aminopropoxy and 2′-fluoro. Similar modifications may also be made at other positions on an oligonucleotide or other nucleobase oligomer, particularly the 3′ position of the sugar on the 3′ terminal nucleotide. Nucleobase oligomers may also have sugar mimetics such as cyclobutyl moieties in place of the pentofuranosyl sugar. Representative United States patents that teach the preparation of such modified sugar structures include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; and 5,700,920, each of which is herein incorporated by reference in its entirety.

In other nucleobase oligomers, both the sugar and the internucleoside linkage, i.e., the backbone, are replaced with novel groups. The nucleobase units are maintained for hybridization with a Glut1 nucleic acid molecule. Methods for making and using these nucleobase oligomers are described, for example, in “Peptide Nucleic Acids (PNA): Protocols and Applications” Ed. P. E. Nielsen, Horizon Press, Norfolk, United Kingdom, 1999. Representative United States patents that teach the preparation of PNAs include, but are not limited to, U.S. Pat. Nos. 5,539,082; 5,714,331; and 5,719,262, each of which is herein incorporated by reference. Further teaching of PNA compounds can be found in Nielsen et al., Science, 1991, 254, 1497-1500.

In other embodiments, a single stranded modified nucleic acid molecule (e.g., a nucleic acid molecule comprising a phosphorothioate backbone and 2′-O-Me sugar modifications is conjugated to cholesterol. Such conjugated oligomers are known as “antagomirs.” Methods for silencing Glut1 polynucleotides in vivo with antagomirs are described, for example, in Krutzfeldt et al., Nature 438: 685-689.

### Glut1 Polynucleotides

In general, the invention includes any nucleic acid sequence encoding a glut1 polynucleotide of as well as nucleic acid molecules containing at least one strand that hybridizes with a nucleic acid sequence of (e.g., an inhibitory nucleic acid molecule, such as an antisense molecule, a dsRNA, siRNA, or shRNA). The inhibitory nucleic acid molecules of the invention can be between 8 and 45 nucleotides in length. In some embodiments, the inhibitory nucleic acid molecules of the invention comprises 8, 10, 15, 16, 17, 18, 19, 20, 21, 22, 23, 24, 25, 26, 27, 28, 29, 30, 31, 32, 33, 34, 35, 45, or complementary nucleotide residues. In yet other embodiments, the antisense molecules are 60%, 65%, 70%, 75%, 80%, 85%, 90%, 95%, 96%, 97%, 98%, 99% complementary to the target sequence. An isolated nucleic acid molecule can be manipulated using recombinant DNA techniques well known in the art. Thus, a nucleotide sequence contained in a vector in which 5′ and 3′ restriction sites are known, or for which polymerase chain reaction (PCR) primer sequences have been disclosed, is considered isolated, but a nucleic acid sequence existing in its native state in its natural host is not. An isolated nucleic acid may be substantially purified, but need not be. For example, a nucleic acid molecule that is isolated within a cloning or expression vector may comprise only a tiny percentage of the material in the cell in which it resides. Such a nucleic acid is isolated, however, as the term is used herein, because it can be manipulated using standard techniques known to those of ordinary skill in the art.

### Delivery of Nucleobase Oligomers

Naked oligonucleotides are capable of entering cells and inhibiting the expression of a glut1 polynucleotide. Nonetheless, it may be desirable to utilize a formulation that aids in the delivery of an inhibitory nucleic acid molecule or other nucleobase oligomers to cells (see, e.g., U.S. Pat. Nos. 5,656,611, 5,753,613, 5,785,992, 6,120,798, 6,221,959, 6,346,613, and 6,353,055, each of which is hereby incorporated by reference).

### Polynucleotide Therapy

Polynucleotide therapy featuring a polynucleotide encoding an inhibitory nucleic acid molecule or analog thereof that targets a glut1 polynucleotide is another therapeutic approach for treating or preventing a diabetic complication in a subject. Expression vectors encoding inhibitory nucleic acid molecules can be delivered to cells of a subject having or at risk of developing a diabetic complication. The nucleic acid molecules must be delivered to the cells of a subject in a form in which they can be taken up and are advantageously expressed so that therapeutically effective levels can be achieved.

Methods for delivery of the polynucleotides to the cell according to the invention include using a delivery system such as liposomes, polymers, microspheres, gene therapy vectors, and naked DNA vectors.

Transducing viral (e.g., retroviral, adenoviral, lentiviral and adeno-associated viral) vectors can be used for somatic cell gene therapy, especially because of their high efficiency of infection and stable integration and expression (see, e.g., Cayouette et al., Human Gene Therapy 8:423-430, 1997; Kido et al., Current Eye Research 15:833-844, 1996; Bloomer et al., Journal of Virology 71:6641-6649, 1997; Naldini et al., Science 272:263-267, 1996; and Miyoshi et al., Proc. Natl. Acad. Sci. U.S.A. 94:10319, 1997).

For example, a polynucleotide encoding an inhibitory nucleic acid molecule can be cloned into a retroviral vector and expression can be driven from its endogenous promoter, from the retroviral long terminal repeat, or from a promoter specific for a target cell type of interest. Other viral vectors that can be used include, for example, a vaccinia virus, a bovine papilloma virus, or a herpes virus, such as Epstein-Ban Virus (also see, for example, the vectors of Miller, Human Gene Therapy 15-14, 1990; Friedman, Science 244:1275-1281, 1989; Eglitis et al., BioTechniques 6:608-614, 1988; Tolstoshev et al., Current Opinion in Biotechnology 1:55-61, 1990; Sharp, The Lancet 337:1277-1278, 1991; Cornetta et al., Nucleic Acid Research and Molecular Biology 36:311-322, 1987; Anderson, Science 226:401-409, 1984; Moen, Blood Cells 17:407-416, 1991; Miller et al., Biotechnology 7:980-990, 1989; Le Gal La Salle et al., Science 259:988-990, 1993; and Johnson, Chest 107:77S-83S, 1995). Retroviral vectors are particularly well developed and have been used in clinical settings (Rosenberg et al., N. Engl. J. Med 323:370, 1990; Anderson et al., U.S. Pat. No. 5,399,346).

Non-viral approaches can also be employed for the introduction of an inhibitory nucleic acid molecule therapeutic to a cell of a patient diagnosed as having diabetes. For example, an inhibitory nucleic acid molecule that targets a Glut1 polynucleotide of can be introduced into a cell by administering the nucleic acid in the presence of lipofection (Feigner et al., Proc. Natl. Acad. Sci. U.S.A. 84:7413, 1987; Ono et al., Neuroscience Letters 17:259, 1990; Brigham et al., Am. J. Med. Sci. 298:278, 1989; Staubinger et al., Methods in Enzymology 101:512, 1983), asialoorosomucoid-polylysine conjugation (Wu et al., Journal of Biological Chemistry 263:14621, 1988; Wu et al., Journal of Biological Chemistry 264:16985, 1989), or by micro-injection under surgical conditions (Wolff et al., Science 247:1465, 1990). Preferably the inhibitory nucleic acid molecules are administered in combination with a liposome and protamine.

Gene transfer can also be achieved using non-viral means involving transfection in vitro. Such methods include the use of calcium phosphate, DEAE dextran, electroporation, and protoplast fusion. Liposomes can also be potentially beneficial for delivery of DNA into a cell.

Inhibitory nucleic acid molecule expression for use in polynucleotide therapy methods can be directed from any suitable promoter (e.g., the human cytomegalovirus (CMV), simian virus 40 (SV40), or metallothionein promoters), and regulated by any appropriate mammalian regulatory element. For example, if desired, enhancers known to preferentially direct gene expression in specific cell types can be used to direct the expression of a nucleic acid. The enhancers used can include, without limitation, those that are characterized as tissue- or cell-specific enhancers.

For any particular subject, the specific dosage regimes should be adjusted over time according to the individual need and the professional judgment of the person administering or supervising the administration of the compositions.

### Pharmaceutical Compositions

As reported herein, Glut1 is present in tissues that are susceptible to diabetes. Accordingly, the invention provides compositions that inhibit the expression or activity of Glut1, as well as methods of using such compositions for the prevention or treatment of diabetes complications. Accordingly, the invention provides therapeutic compositions that decrease the expression of a glut1 polynucleotide to treat or prevent a diabetic complication. In one embodiment, the present invention provides a pharmaceutical composition comprising an inhibitory nucleic acid molecule (e.g., an antisense, siRNA, or shRNA polynucleotide) that decreases the expression of Glut1.

In one embodiment, the invention provides pharmaceutical compositions comprising an siRNA that targets a glut1 polynucleotide. Exemplary target sequences and siRNAs follow:

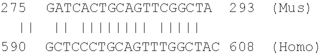

siRNA #14

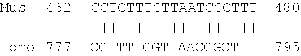

siRNA #15

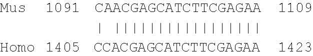

siRNA #16

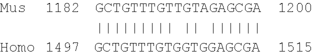

In a preferred embodiment, the siRNA is #14 bp 777 ccttttcgttaacc gcttt 795.

In other preferred embodiments, the pharmaceutical composition comprises an expression vector encoding an inhibitory nucleic acid molecule of the invention.

If desired, the inhibitory nucleic acid molecule is administered in combination with a agent useful for treating a diabetic complication. Polynucleotides of the invention may be administered as part of a pharmaceutical composition. The compositions should be sterile and contain a therapeutically effective amount of the polypeptides or nucleic acid molecules in a unit of weight or volume suitable for administration to a subject.

An inhibitory nucleic acid molecule of the invention, or other negative regulator of a Glut1 polynucleotide may be administered within a pharmaceutically-acceptable diluent, carrier, or excipient, in unit dosage form. Conventional pharmaceutical practice may be employed to provide suitable formulations or compositions to administer the compounds to patients suffering from diabetes, a complication of diabetes, or a pre-diabetic condition. Administration may begin before the patient is symptomatic. Any appropriate route of administration may be employed, for example, administration may be parenteral, intravenous, intraarterial, subcutaneous, intraocular, intramuscular, intracranial, intraorbital, ophthalmic, intraventricular, intrahepatic, intracapsular, intrathecal, intracisternal, intraperitoneal, intranasal, aerosol, suppository, or oral administration. For example, therapeutic formulations may be in the form of liquid solutions or suspensions; for oral administration, formulations may be in the form of tablets or capsules; and for intranasal formulations, in the form of powders, nasal drops, or aerosols.

Methods well known in the art for making formulations are found, for example, in “Remington: The Science and Practice of Pharmacy” Ed. A. R. Gennaro, Lippincourt Williams & Wilkins, Philadelphia, Pa., 2000. Formulations for parenteral administration may, for example, contain excipients, sterile water, or saline, polyalkylene glycols such as polyethylene glycol, oils of vegetable origin, or hydrogenated napthalenes. Biocompatible, biodegradable lactide polymer, lactide/glycolide copolymer, or polyoxyethylene-polyoxypropylene copolymers may be used to control the release of the compounds. Other potentially useful parenteral delivery systems for inhibitory nucleic acid molecules include ethylene-vinyl acetate copolymer particles, osmotic pumps, implantable infusion systems, and liposomes. Formulations for inhalation may contain excipients, for example, lactose, or may be aqueous solutions containing, for example, polyoxyethylene-9-lauryl ether, glycocholate and deoxycholate, or may be oily solutions for administration in the form of nasal drops, or as a gel.

The formulations can be administered to human patients in therapeutically effective amounts (e.g., amounts which prevent, eliminate, or reduce a pathological condition) to provide therapy for a diabetic complication. The preferred dosage of a nucleobase oligomer of the invention is likely to depend on such variables as the type and extent of the disorder, the overall health status of the particular patient, the formulation of the compound excipients, and its route of administration.

With respect to a subject having a diabetic complication disease or disorder, an effective amount is sufficient to prevent, stabilize, slow, or reduce the severity of the complication. Generally, doses of active polynucleotide compositions of the present invention would be from about 0.01 mg/kg per day to about 1000 mg/kg per day. It is expected that doses ranging from about 50 to about 2000 mg/kg will be suitable. Lower doses will result from certain forms of administration, such as intravenous administration. In the event that a response in a subject is insufficient at the initial doses applied, higher doses (or effectively higher doses by a different, more localized delivery route) may be employed to the extent that patient tolerance permits. Multiple doses per day are contemplated to achieve appropriate systemic levels of an antisense targeting.

### Therapy

Therapy may be provided wherever diabetes therapy is performed: at home, the doctor's office, a clinic, a hospital's outpatient department, or a hospital. Treatment generally begins at a hospital so that the doctor can observe the therapy's effects closely and make any adjustments that are needed. The duration of the therapy depends on the complication being treated, the age and condition of the patient, the stage and type of the patient's disease, and how the patient's body responds to the treatment. Drug administration may be performed at different intervals (e.g., daily, weekly, or monthly).

### Patient Monitoring

The disease state or treatment of a patient having a diabetic complication can be monitored using the methods and compositions of the invention. Such monitoring may be useful, for example, in assessing the efficacy of a particular drug in a patient. Therapeutics that reduce glucose and glycohemoglobin levels in red blood cells are taken as particularly effective for the treatment of prevention of a diabetic complication.

Agents (e.g., inhibitory nucleic acids, such as siRNAs, antisense molecules, shRNAs) of the invention can, for example, be administered by injection, intraocularly, intravitreally, subretinal, intravenously, intraarterially, subdermally, intraperitoneally, intramuscularly, or subcutaneously; or orally, buccally, nasally, transmucosally, directly to a diseases organ by catheter, topically, or in an ophthalmic preparation, with a dosage ranging from about 0.001 to about 100 mg/kg of body weight, or according to the requirements of the particular drug and more preferably from 0.5-10 mg/kg of body weight. It is understood that when a compound is delivered directly to the eye, considerations such as body weight have less bearing on the dose. For ocular administration, especially subretinal administration, the total volume for administration is of substantial concern with the preferred dosage being in the smallest volume possible for dosing. For administration of viral particles, dosages are typically provided by number of virus particles (or viral genomes) and effective dosages would range from about 10³ to 10¹² particles, 10⁵ to 10¹¹ particles, 10⁶ to 10¹⁰ particles, 10⁸ to 10¹¹ particles, or 10⁹ to 10¹⁰ particles. The effective dose can be the number of particles delivered for each expression construct to be delivered when different expression constructs encoding different genes are administered separately. In alternative embodiment, the effective dose can be the total number of particles administered, of one or more types. The methods herein contemplate administration of an effective amount of compound or compound composition to achieve the desired or stated effect.

### Polynucleotide Delivery

Compositions and methods for polynucleotide delivery to various organs and cell types in the body are known to those of skill in the art. Such compositions and methods are provided, for example in U.S. Pat. Nos. 7,459,153; 7,041,284; 6,849,454; 6,410,011; 6,027,721; and 5,705,151, all of which are incorporated herein by reference. Expression constructs provided in the listed patents and any other known expression constructs for gene delivery can be used in the compositions and methods of the invention.

### Polynucleotide Delivery to the Eye

The eye has unique advantages as a target organ for the development of novel therapies and is often regarded as a valuable model system for gene therapy. It is a relatively small target organ with highly compartmentalized anatomy in which it is possible to deliver small volumes of expression vectors for gene delivery, in the context of a viral particle, as nucleic acid alone, or nucleic acid complexed with other agents. It is possible to obtain precise, efficient, and stable transduction of a variety of ocular tissues with attenuated immune responses due to the immune privilege nature of the eye. The risks of systemic side effects for eye procedures are minimal. Further, if only one eye is treated, the untreated eye may serve as a useful control. Gene therapy offers a potentially powerful modality for the management of both rare and common complex acquired disorders (Banibridge, 2008. Gene Therapy 15:633-634, incorporated herein by reference).

Compositions and methods provided herein include the use of gene delivery to the eye for expression of a peroxidase, a superoxide dismutase, or both. In three stage I clinical trials for the treatment of ocular disease, specifically Leber Congenital Amaurosis, an incurable retinal degeneration that causes severe vision loss, gene delivery using an adenoassociated virus administered subretinally has been demonstrated to be safe. Moreover, as a secondary outcome, improvement in visual function was observed in seven of the first nine treated patients. (Bainbridge, 2008. N Engl J Med. 358:2231-9; Maguire, 2008. N Engl J Med. 358:2240-8; Miller, 2008. N Engl J Med. 358:2282-4; Hauswirth, 2008. Hum Gene Ther. September 7; [Epub ahead of print], each incorporated herein by reference) These data demonstrate that gene delivery can be effective for the treatment of an otherwise incurable ocular disease.

The viral vectors used in each of the studies demonstrate that various gene therapy viral vector designs can be useful for gene deliver. Methods of viral vector design and generation are well known to those of skill in the art, and methods of preparation of viral vectors can be performed by any of a number of companies as demonstrated below. Expression constructs provided herein can be inserted into any of the exemplary viral vectors listed below. Alternatively, viral vectors can be generated base on the examples provided below.

For example, in the Bainbridge study, the tgAAG76 vector, a recombinant adeno-associated virus vector of serotype 2 was used for gene delivery. The vector contains the human RPE65 coding sequence driven by a 1400-bp fragment of the human RPE65 promoter and terminated by the bovine growth hormone polyadenylation site, as described elsewhere. The vector was produced by Targeted Genetics Corporation according to Good Manufacturing Practice guidelines with the use of a B50 packaging cell line, an adenovirus-adeno-associated virus hybrid shuttle vector containing the tgAAG76 vector genome, and an adenovirus 5 helper virus. The vector was filled in a buffered saline solution at a titer of 1×10¹¹ vector particles per milliliter and frozen in 1-ml aliquots at −70° C.

Vectors of the invention preferably include AAV2/9 for our application. and methods of making them are known in the art. See, for example, Campbell et al., Retinal Degeneration Methods in Molecular Biology Volume 935, 2013, pp 371-380 and Campbell et al. EMBO Mol Med 3, 235-245, which shows that AAV2/9 targets retinal endothelial cells.

Maguire used the recombinant AAV2.hRPE65v2 viral vector which is a replication-deficient AAV vector containing RPE65 cDNA that has been documented to provide long-term, sustained (>7.5 years, with ongoing observation) restoration of visual function in a canine model of LCA2 after a single subretinal injection of AAV2.RPE65. The cis plasmid used to generate AAV2.RPE65 contains the kanamycin-resistance gene, and the transgene expression cassette contains a hybrid chicken β-actin (CBA) promoter comprising the cytomegalovirus immediate early enhancer (0.36 kb), the proximal CBA promoter (0.28 kb), and CBA exon 1 flanked by intron 1 sequences (0.997 kb). To include a Kozak consensus sequence at the translational start site, the sequence surrounding the initiation codon was modified from GCCGCATGT in the original vector to CCACCATGT. The virus was manufactured by The Center for Cellular and Molecular Therapeutics after triple transfection of HEK293 cells and was isolated and purified by microfluidization, filtration, cationexchange chromatography (POROS 50HS; GE Healthcare, Piscataway, N.J.), density gradient ultracentrifugation and diafiltration in PBS. This combination provides optimal purity of the AAV vector product, including efficient removal of empty capsids and residual cesium chloride. A portion of the product was supplemented with PF68 NF Prill Poloxamer 188 (PF68; BASF, Ludwigshafen, Germany) to prevent subsequent losses of vector to product contact surfaces. The purified virus, with or without PF68, was then passed through a 0.22-μm filter using a sterile 60-ml syringe and syringe filter, and stored frozen (−80° C.) in sterile tubes until use. An injection of 1.5×10¹⁰ vector genome of AAV2.hRPE65v2 in a volume of 150 μl of phosphate-buffered saline supplemented with Pluronic F-68 NF Prill Poloxamer 188 was administered into the subretinal space,

The viral vector used by Hauswirth was a recombinant adeno-associated virus serotype 2 (rAAV2) vector, altered to carry the human RPE65 gene (rAAV2-CB^{SB}-hRPE65), that had been previously demonstrated to restore vision in animal models with RPE65 deficiency. The viral vector includes, in order from 5′ to 3′, an inverted terminal repeat sequence (ITR), a CMV immediate early enhancer, a β-actin promoter, β-actin exon 1, β-actin intron 1, β-actin exon 3, wild-type human RPE65 sequence, SV40 poly(A) sequence, and an inverted terminal repeat. The RPE65-LCA viral vector was delivered by subretinal injection (5.96×10¹⁰ vector genomes in 150 μl).

Further AAV vectors are provided in the review by Rolling 2004 (Gene Therapy 11: S26-S32, incorporated herein by reference). Hybrid AAV viral vectors, including AAV 2/4 and AAV2/5 vectors are provided, for example, by U.S. Pat. No. 7,172,893 (incorporated herein by reference). Such hybrid virus particles include a parvovirus capsid and a nucleic acid having at least one adeno-associated virus (AAV) serotype 2 inverted terminal repeat packaged in the parvovirus capsid. However, the serotypes of the AAV capsid and said at least one of the AAV inverted terminal repeat are different. For example, a hybrid AAV2/5 virus in which a recombinant AAV2 genome (with AAV2 ITRs) is packaged within a AAV Type 5 capsid.

Self-complementary AAV (scAAV) vectors have been developed to circumvent rate-limiting second-strand synthesis in single-stranded AAV vector genomes and to facilitate robust transgene expression at a minimal dose (Yokoi, 2007. IOVS. 48:3324-3328, incorporated herein by reference). Self-complementary AAV-vectors were demonstrated to provide almost immediate and robust expression of the reporter gene inserted in the vector. Subretinal injection of 5×10⁸ viral particles (vp) of scAAV.CMV-GFP resulted in green fluorescent protein (GFP) expression in almost all retinal pigment epithelial (RPE) cells within the area of the small detachment caused by the injection by 3 days and strong, diffuse expression by 7 days. Expression was strong in all retinal cell layers by days 14 and 28. In contrast, 3 days after subretinal injection of 5×10⁸ vp of ssAAV.CMV-GFP, GFP expression was detectable in few RPE cells. Moreover, the ssAAV vector required 14 days for the attainment of expression levels comparable to those observed using scAAV at day 3. Expression in photoreceptors was not detectable until day 28 using the ssAAV vector. The use of the scAAV vector in the gene delivery methods of the invention can allow for prompt and robust expression from the expression construct. Moreover, the higher level of expression from the scAAV viral vectors can allow for delivery to of the viral particles intravitreally rather than subretinally.

Various recombinant AAV viral vectors have been designed including one or more mutations in capsid proteins or other viral proteins. It is understood that the use of such modified AAV viral vectors falls within the scope of the instant invention.

Adenoviral vectors have also been demonstrated to be useful for gene delivery. For example, Mori et al (2002. IOVS, 43:1610-1615, incorporated herein by reference) discloses the use of an adenoviral vector that is an E-1 deleted, partially E-3 deleted type 5 Ad in which the transgene (green fluorescent protein) is driven by a CMV promoter. Peak expression levels were demonstrated upon injection of 10⁷ to 10⁸ viral particles, with subretinal injection providing higher levels of expression than intravitreal injection.

Efficient non-viral ocular gene transfer was demonstrated by Farjo et al. (2006, PLoS 1:e38, incorporated herein by reference) who used compacted DNA nanoparticles as a system for non-viral gene transfer to ocular tissues. As a proof of concept, the pZEEGFP5.1 (5,147 bp) expression construct that encodes the enhanced green fluorescent protein (GFP) cDNA transcriptionally-controlled by the CMV immediate-early promoter and enhancer was used. DNA nanoparticles were formulated by mixing plasmid DNA with CK3OPEG10K, a 30-mer lysine peptide with an N-terminal cysteine that is conjugated via a maleimide linkage to 10 kDa polyethylene glycol using known methods. Nanoparticles were concentrated up to 4 mg/ml of DNA in saline. The compacted DNA was delivered at a 0.6 μg dose to the vitreal cavity. GFP expression was observed in the lens, retina, and pigment epithelium/choroid/sclera by PCR and microscopy.

Further, a number of patents have been issued for methods of ocular gene transfer including, but not limited to, U.S. Pat. No. 7,144,870 which provides methods of hyaluronic acid mediated adenoviral transduction; U.S. Pat. Nos. 7,122,181 and 6,555,107 which provide lentiviral vectors and their use to mediate ocular gene delivery; U.S. Pat. No. 6,106,826 which provides herpes simplex viral vectors and their use to mediate ocular gene delivery; and U.S. Pat. No. 5,770,580 which provides DNA expression vectors and their use to mediate ocular gene delivery. Each of these patents is incorporated herein by reference.

### Self-Complementary Adenoviral Vectors

Under normal circumstances, AAV packages a single-stranded DNA molecule of up to 4800 nucleotides in length. Following infection of cells by the virus, the intrinsic molecular machinery of the cell is required for conversion of single-stranded DNA into double stranded form. The double-stranded form is then capable of being transcribed, thereby allowing expression of the delivered gene to commence. It has been shown in a number of cell and tissue types that second strand synthesis of DNA by the host cell is the rate-limiting step in expression. By virtue of already being packaged as a double stranded DNA molecule, self-complementary AAV (scAAV) bypasses this step, thereby greatly reducing the time to onset of gene expression.

Self-complementary AAV is generated through the use of vector plasmid with a mutation in one of the terminal resolution sequences of the AAV virus. This mutation leads to the packaging of a self-complementary, double-stranded DNA molecule covalently linked at one end. Vector genomes are required to be approximately half genome size (2.4 KB) in order to package effectively in the normal AAV capsid. Because of this size limitation, large promoters are unsuitable for use with scAAV. Most broad applications to date have used the cytomegalovirus immediate early promoter (CMV) alone for driving transgene expression. However, it has been shown by others that transgene expression with CMV markedly drops off in certain tissue types, such as eye and liver, sometimes as early as two weeks post-injection. A long acting, ubiquitous promoter of small size would be very useful in a scAAV system.

### Nucleic Acid Regulatory Sequences

The invention provides expression constructs that include any regulatory sequences that are functional in the cells in which protein expression is desired, e.g., retinal pigment epithelial (RPE) cells, rod cells, cone cells, etc. For example, cell and tissue specific promoters such as the interphotoreceptor retinoid binding protein (Fei, 1999, J. Biochem. 125:1189-1199, and Liou, 1991, BBRC. 181:159-165, both incorporated herein by reference), cone arrestin promoter (Pickrell, 2004. IOVS. 45:3877-3884, incorporated herein by reference), RPE65 promoter, and cis-Retinaldehyde-binding protein (CRALBP) promoter is a retinal-pigment-epithelium (RPE)-specific promoter (2,265 bp) when administered subretinally in a rAAV vector can be used in the expression constructs of the instant invention. Alternatively, non-tissue specific promoters including viral promoters such as cytomegalovirus (CMV) promoter, and β-actin promoter can be used such as the chicken β-actin (CBA) promoter.

The chimeric CMV-chicken β-actin promoter (CBA) has been utilized extensively as a promoter that supports expression in a wide variety of cells when in rAAV vectors delivered to retina, including in the clinical trials discussed herein. In addition to broad tropism, the present inventors have observed that CBA also has the capacity to promote expression for long periods post infection (Acland, G. M. et al. MoI Then, 2005, 12:1072-1082, incorporated herein by reference). CBA is −1700 base pairs in length, too large in most cases to be used in conjunction with scAAV to deliver cDNAs (over 300 bps pairs in length). CBA is a ubiquitous strong promoter composed of a cytomegalovirus (CMV) immediate-early enhancer (381 bp) and a CBA promoter-exon1-intron1 element (1,352 bp) (Raisler Proc Natl Acad Sci USA. 2002 Jun. 25; 99(13): 8909-8914, incorporated herein by reference). A shortened CBA promoter sequence, the smCBA promoter sequence, has also been described in which the The total size of smCBA is 953 bps versus 1714 bps for full length CBA. The smCBA promoter is described in Mah, et al. 2003 (Hum. Gene Ther. 14:143-152, incorporated herein by reference) and Haire, et al. 2006 (IOVS, 2006, 47:3745-3753, incorporated herein by reference).

Other regulatory sequences for inclusion in expression constructs include poly-A signal sequences, for example SV40 polyA signal sequences. The inclusion of a splice site (i.e., exon flanked by two introns) has been demonstrated to be useful to increase gene expression of proteins from expression constructs.

For viral sequences, the use of viral sequences including inverted terminal repeats, for example in AAV viral vectors can be useful. Certain viral genes can also be useful to assist the virus in evading the immune response after administration to the subject.

In certain embodiments of the invention, the viral vectors used are replication deficient, but contain some of the viral coding sequences to allow for replication of the virus in appropriate cell lines. The specific viral genes to be partially or fully deleted from the viral coding sequence is a matter of choice, as is the specific cell line in which the virus is propagated. Such considerations are well known to those of skill in the art.

### Codon Optimization

Expression construct design and generation can include the use of codon optimization. The degeneracy of the genetic code is well known with more than one nucleotide triplet coding for most of the amino acids, e.g., each leucine, arginine, and serine are encoded by five different codons each. It is possible to design multiple nucleotide sequences that encode a single amino acid sequence. Redesign of a nucleotide sequence without changing the sequence of the polypeptide encoded is well within the ability of those of skill in the art.

### Kits

The present invention also encompasses a finished packaged and labeled pharmaceutical product or laboratory reagent. This article of manufacture includes the appropriate instructions for use in an appropriate vessel or container such as a glass vial or other container that is hermetically sealed. A pharmaceutical product may contain, for example, a compound of the invention in a unit dosage form in a first container, and in a second container, sterile water or adjuvant for injection. Alternatively, the unit dosage form may be a solid suitable for parenteral delivery, particularly intraocular delivery.

As with any pharmaceutical product, the packaging material and container are designed to protect the stability of the product during storage and shipment. Further, the products of the invention include instructions for use or other informational material that advise the physician, technician, or patient on how to appropriately prevent or treat the disease or disorder in question. In other words, the article of manufacture includes instructions indicating or suggesting a dosing regimen including, but not limited to, actual doses, monitoring procedures (e.g. visual acuity testing), and other monitoring information.

Specifically, the invention provides an article of manufacture including packaging material, such as a box, bottle, tube, vial, container, sprayer, needle for intraocular administration, envelope and the like; and at least one unit dosage form of a pharmaceutical agent contained within said packaging material, wherein said pharmaceutical agent comprises a compound of the invention, and wherein said packaging material includes instruction means which indicate that said compound can be used to prevent, manage, treat, and/or ameliorate one or more symptoms associated with oxidative stress associated ocular disease by administering specific doses and using specific dosing regimens as described herein.

### Co-Administration of Compounds

The compositions and methods of the invention can be combined with any other composition(s) and method(s) known or not yet known in the art for the prevention, amelioration, or treatment of diseases associated with oxidative stress.

Other strategies for uses of siRNA, shRNA, antisense, and other agents for the treatment of diseases related to oxidative stress can be envisioned.

The following examples are provided merely as illustrative of various aspects of the invention and shall not be construed to limit the invention in any way.

### EXAMPLES

### Example 1

### Knockdown or Blockade of GLUT1 Reduces Retinal Glucose in Diabetic Mice

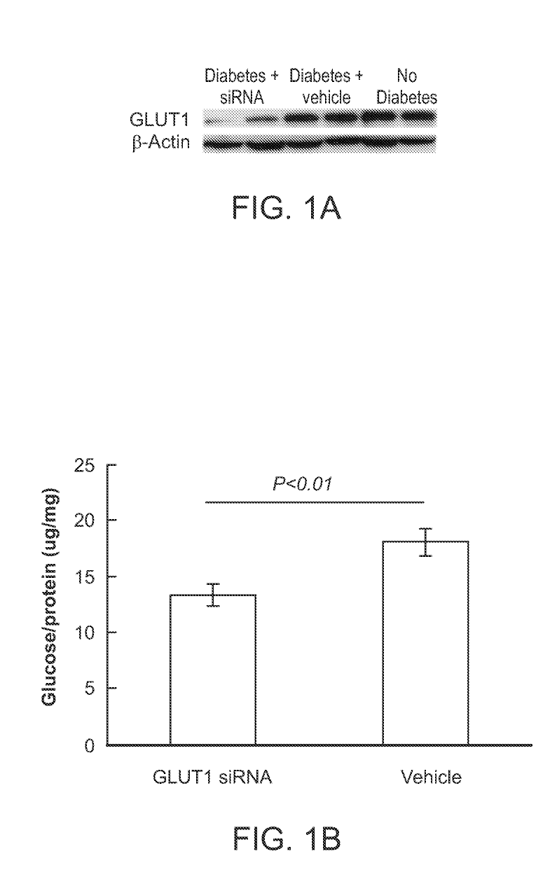

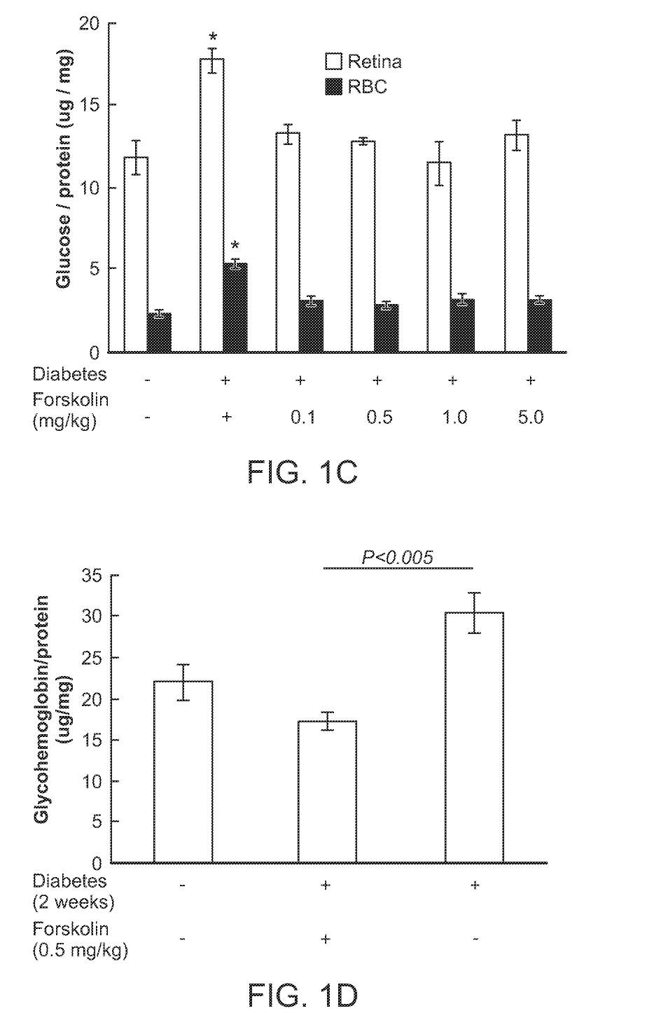

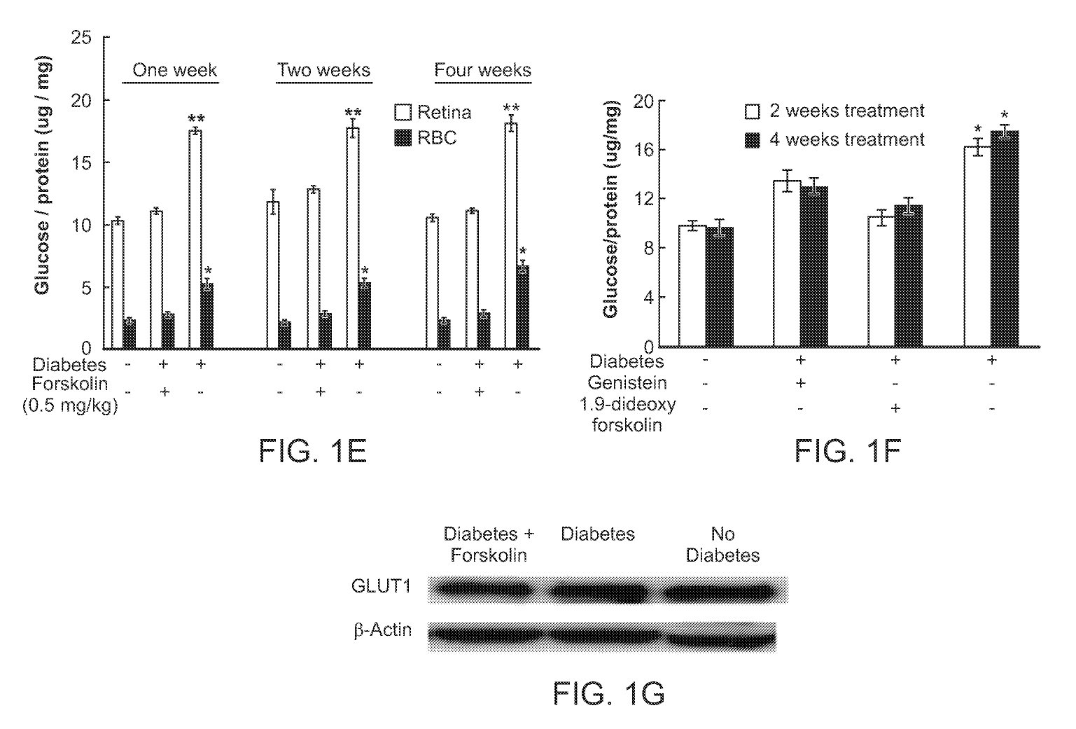

Transport of glucose from blood into the retina through the blood-retinal harrier (BRB) is mediated solely by GLUT1 (Mantych et al., 1993; Kumagai et al., 1994). Without being bound to a particular theory, it was hypothesized that knockdown of GLUT1 with a pool of siRNAs specifically targeting its mRNA could reduce the glucose level in the retinas of mice with streptozotocin-induced diabetes. Mice with sustained hyperglycemia for several weeks after administration of streptozotocin were given an injection of vehicle in one eye and vehicle containing 1 μg of a pool of siRNAs directed against SLC2A1 mRNA which codes for GLUT1 on days 1, 4, and 7 in the other eye. On day 10, immunoblots of retinal homogenates showed strong knockdown of GLUT1 in retinas of siRNA-injected eyes compared to those injected with vehicle or those of nondiabetic mice (FIG. 1A). The glucose concentration in retinal homogenates from siRNA-injected eyes was significantly less than that from vehicle-injected eyes (FIG. 1B).

Forskolin is a diterpene that is best known for its ability to activate adenylate cyclase and increase levels of cyclic AMP (Seamon and Daly, 1981); however, it also binds GLUT1 and allosterically blocks transport of glucose (Kashiwagi et al., 1983; Sergeant and Kim, 1985; Kim et al., 1986; Shanahan et al., 1987). Compared to untreated mice with streptozotocin-induced diabetes for 2 weeks, those treated with daily IP injections of forskolin ranging from 0.1 to 5 mg/kg, had a significant reduction in the level of glucose in the retina (FIG. 1C). As is true for the BRB, GLUT1 is the sole glucose transporter for RBCs and thus glucose levels in RBCs may provide a peripheral marker that could reflect glucose levels in the retina. This was found to be the case for forskolin treatment of diabetics, because untreated diabetics had high glucose levels in RBCs that was significantly reduced by forskolin treatment for 2 weeks (FIG. 1C). Also, after 2 weeks of diabetes, there was an increase in glycosylated hemoglobin in RBCs that was prevented by IP injections of 0.5 mg/kg/day of forskolin (FIG. 1D). One week after the onset of diabetes, mean blood glucose was high in forskolin-treated or untreated diabetics and was similarly high after 2 or 4 weeks of diabetes (FIG. 1E). Compared to non-diabetics, glucose levels were significantly elevated in RBCs and retina after 1 week of diabetes with further elevation over time, but diabetics treated with 0.5 mg/kg/day of forskolin had no difference in retinal or RBC glucose levels compared to non-diabetics at 1, 2, or 4 weeks of diabetes.

An analog of forskolin, 1,9-dideoxyforskolin, lacks adenylate cyclase-stimulating activity, but blocks GLUT1-mediated glucose transport (Joost et al., 1988). Treatment of diabetic mice with 0.1 mg/kg/day of 1,9-dideoxyforskolin significantly reduced retinal glucose levels compared to untreated diabetics indicating that elevation of cAMP is not needed for the glucose transport-blocking activity of forskolin (FIG. 1F). Genistein is an isoflavone that inhibits tyrosine kinases by blocking the ATP binding site (Akiyama et al., 1987) and it also binds to the ATP binding site of GLUT1 and suppresses glucose transport (Vera et al., 2001). Treatment of diabetic mice with 50 mg/kg/day of genistein significantly reduced retinal glucose levels compared to untreated diabetics (FIG. 1F). Unlike siRNA directed against GLUT1, forskolin did not reduce GLUT1 protein levels in the retina (FIG. 1G).

### Example 2

### Blockade of GLUT1 Reduces Biomarkers of Diabetic Retinopathy

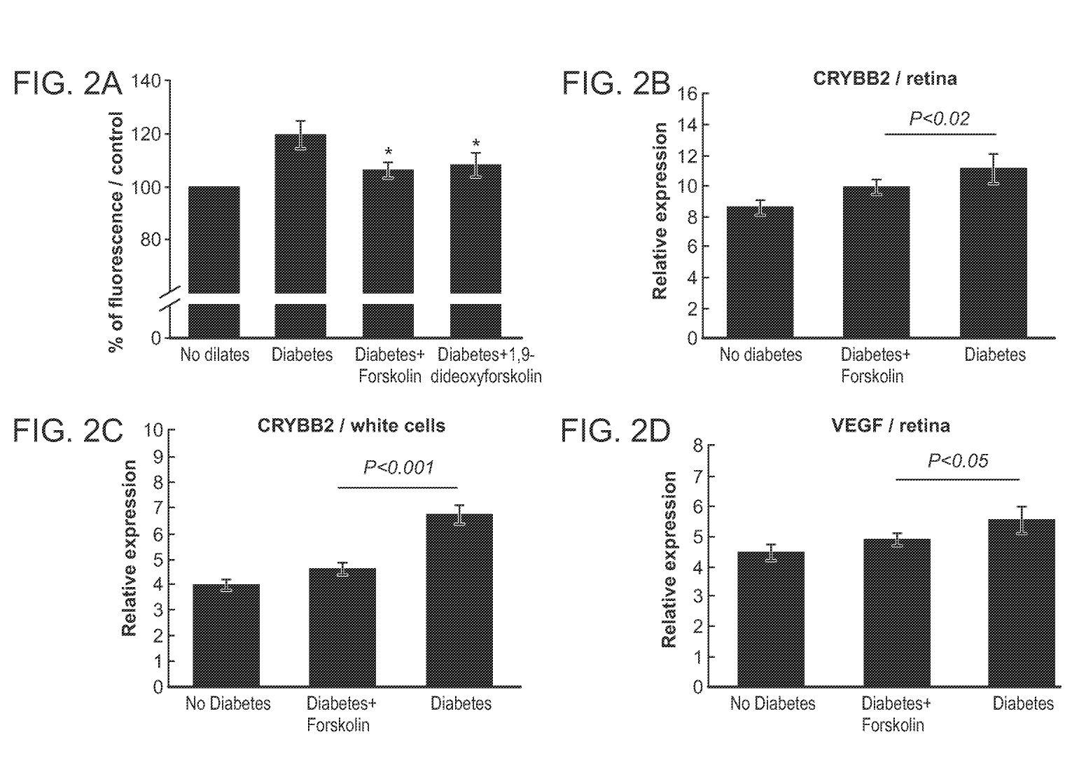

While the mechanism(s) by which high glucose levels in the retina lead to diabetic retinopathy is uncertain, there is strong evidence suggesting that oxidative stress plays a role (Du et al., 2000; Nishikawa et al., 2000). One of the earliest consequences of high intracellular glucose is excessive production of superoxide radicals which activates several pathologic processes that contribute to the development of diabetic complications (for reviews, see Kowluru and Chan, 2007; Giacco and Brownlee, 2010). In the presence of superoxide radicals, hydroethidine is converted to ethidium which binds DNA and fluoresces (Pietch et al., 2003). After 1 month of diabetes, hydroethidine-induced fluorescence was increased in retinal homogenates and that increase was significantly blocked by daily injections of 0.5 mg/kg of forskolin or 0.1 mg/kg of 1,9-dideoxyforskolin (FIG. 2A).

Chaperone proteins are increased to compensate for oxidative stress and have been shown to be up-regulated early in diabetic retina and the up-regulation is significantly reduced by treatment with insulin (Kumar et al., 2005; Fort et al., 2009; Losiewicz and Fort, 2011). CRYBB2 mRNA, which codes for β2 crystallin (Andley, 2007), was significantly elevated in the retina after 1 month of diabetes and the elevation was significantly suppressed by daily injections of 0.5 mg/kg of forskolin (FIG. 2B). CRYBB2 mRNA was also increased in WBCs after 1 month of diabetes and that too was blocked by forskolin providing a peripheral biomarker of this effect in the retina (FIG. 2C).

VEGF is a stimulator of neovascularization that plays an important role in the advanced stages of diabetic retinopathy; however, modest oxidative-stress induced increases in Vegf mRNA have been reported early in diabetic retinopathy and have been postulated to play a role in the development of background diabetic retinopathy (Obrosova et al., 2001; Yamagishi et al., 2006; Goto et al., 2008). There was a small increase in Vegf mRNA in the retina after 1 month of diabetes that was blocked in forskolin-treated diabetics (FIG. 2D).

### Example 3

### Glucose Levels are not Increased in Brain in Diabetic Mice and are not Reduced by Blockade of GLUT1

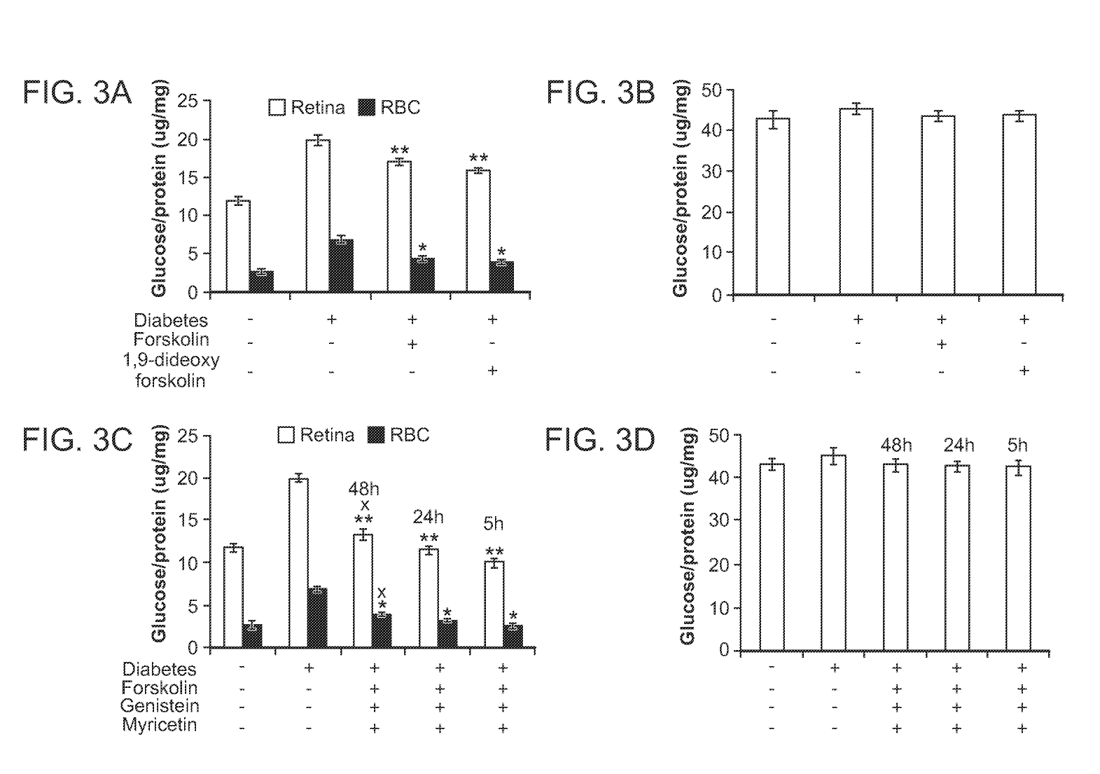

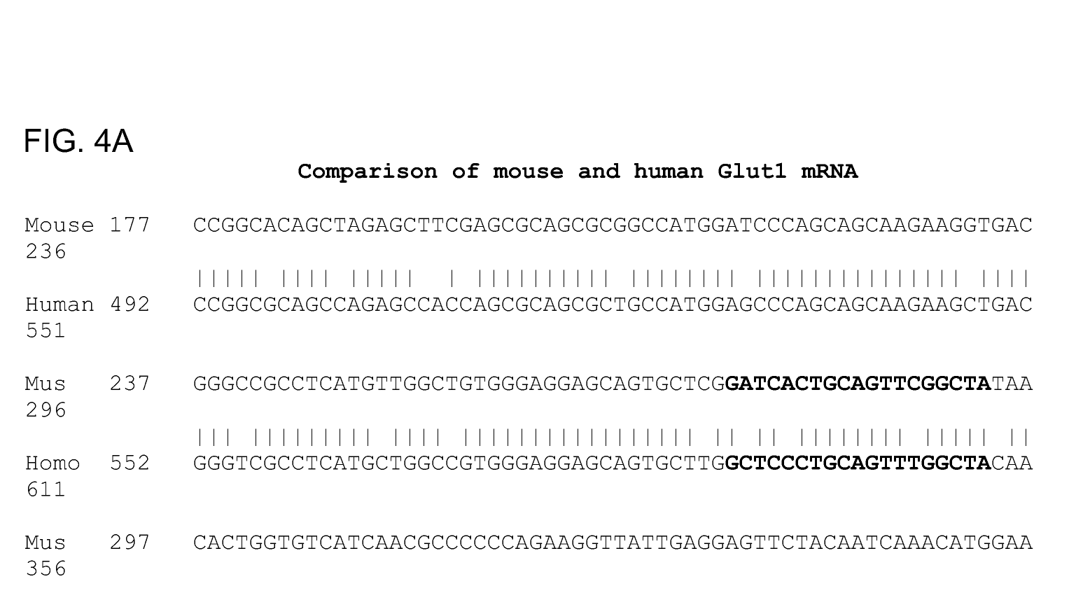

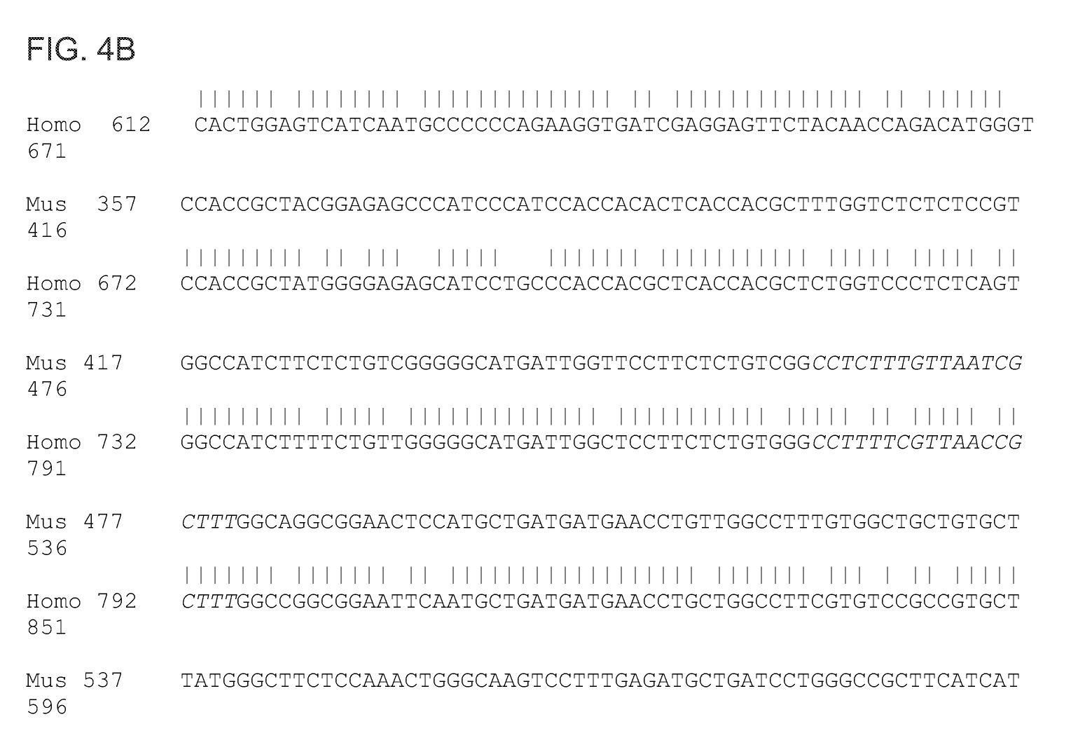

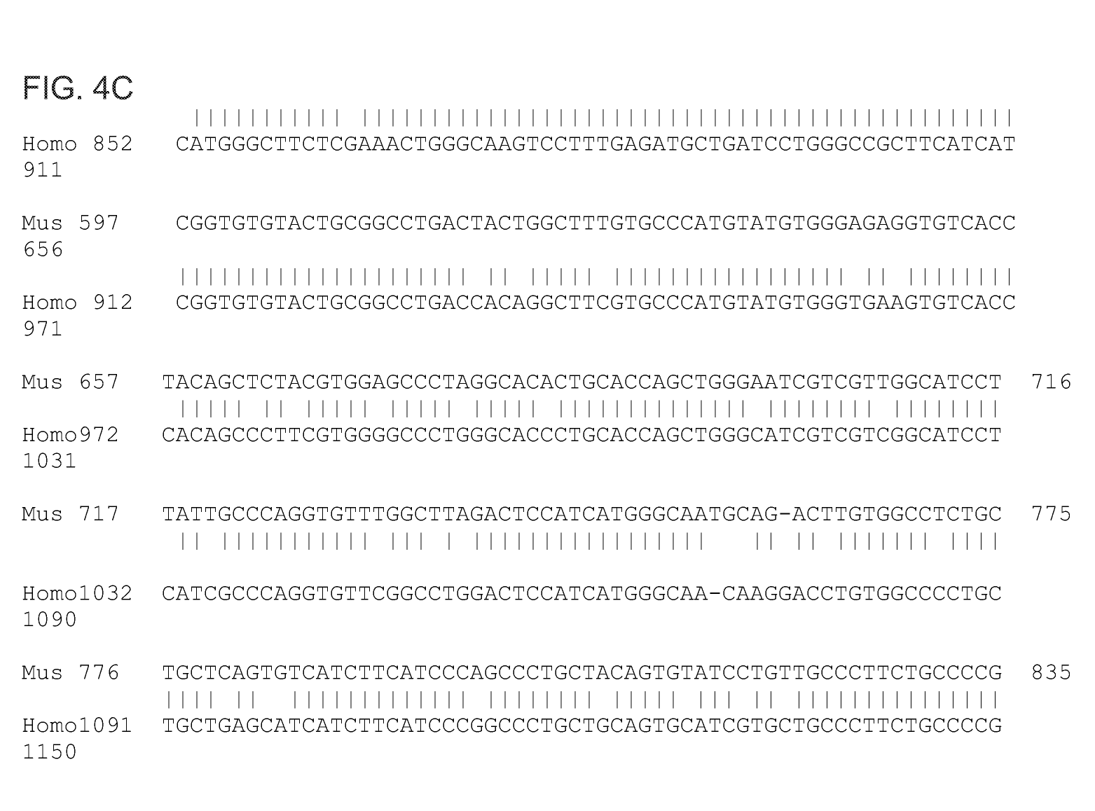

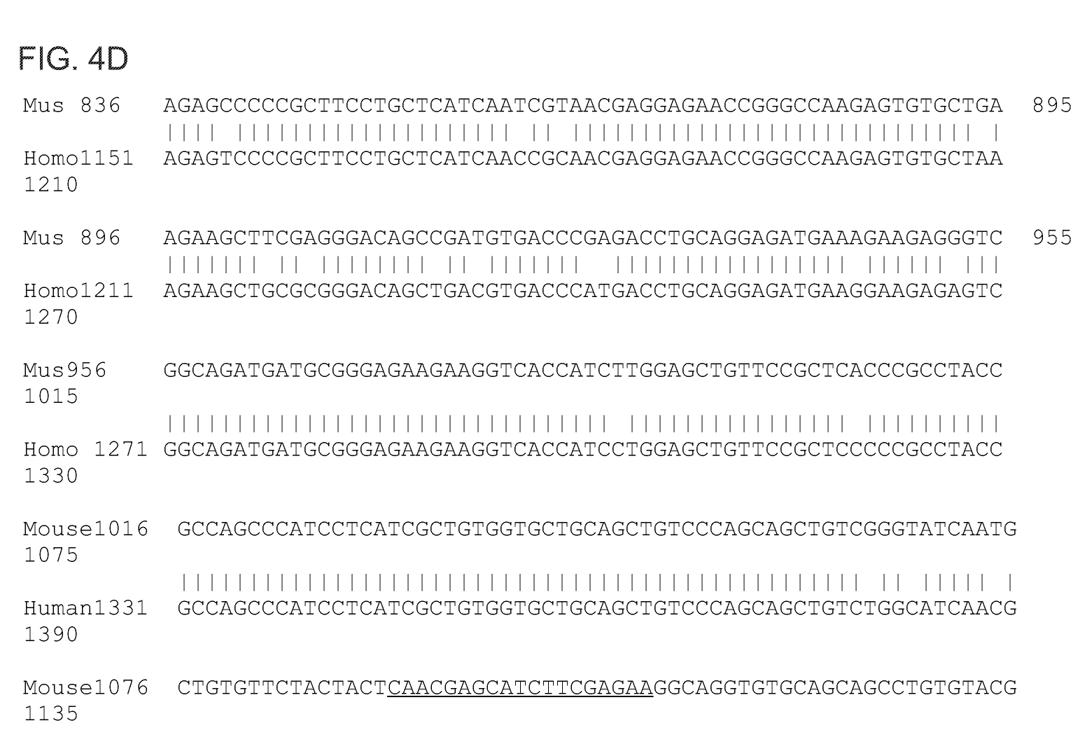

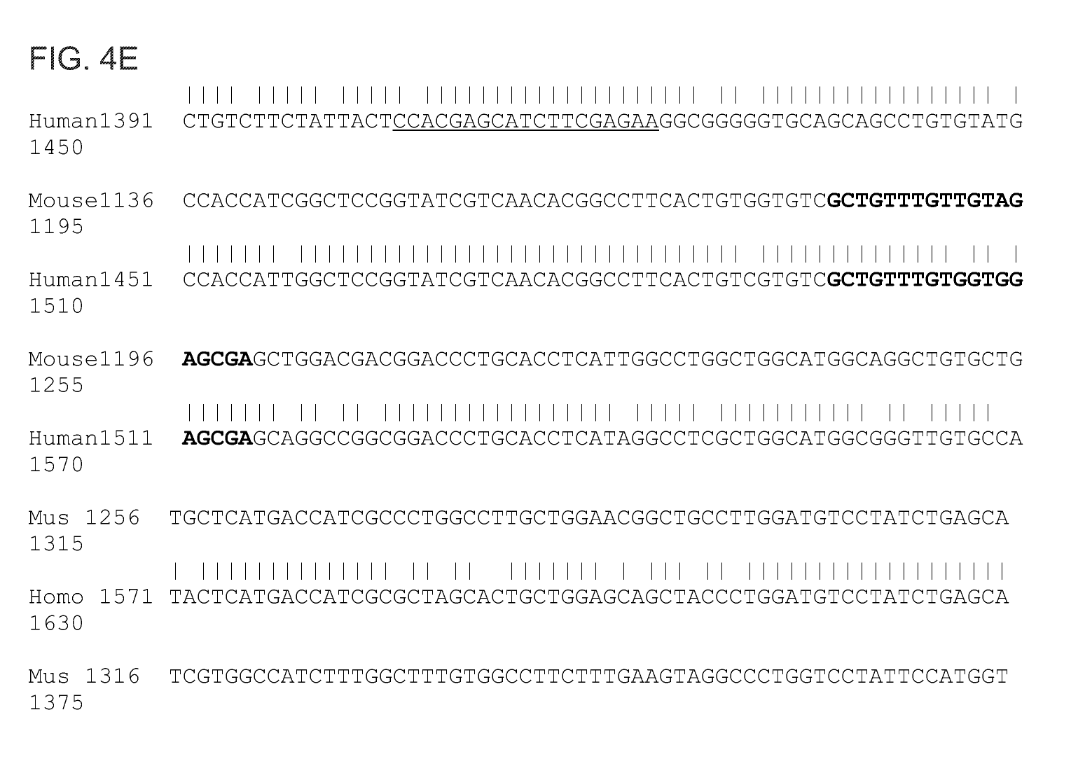

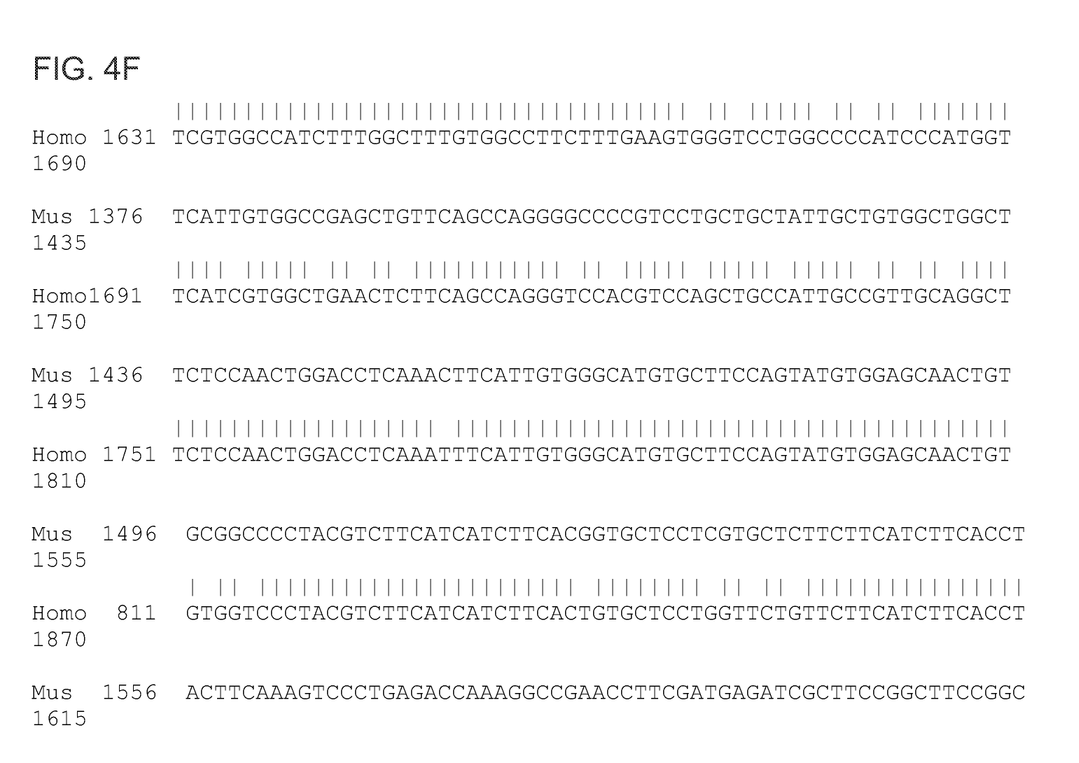

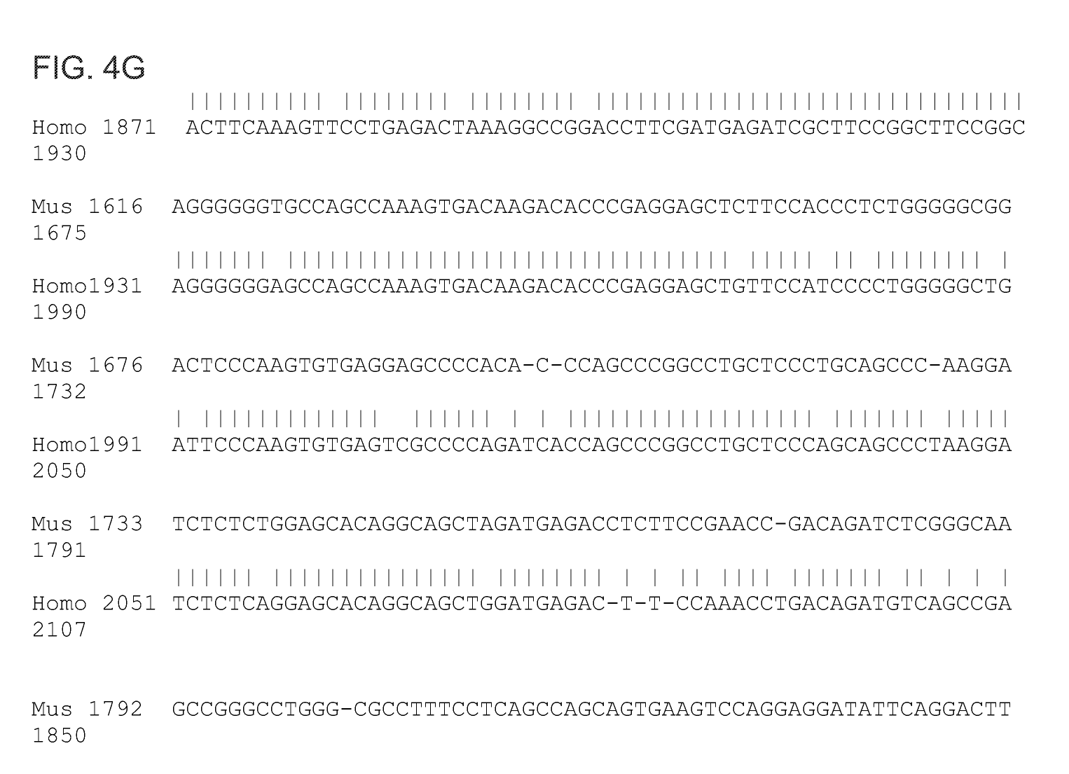

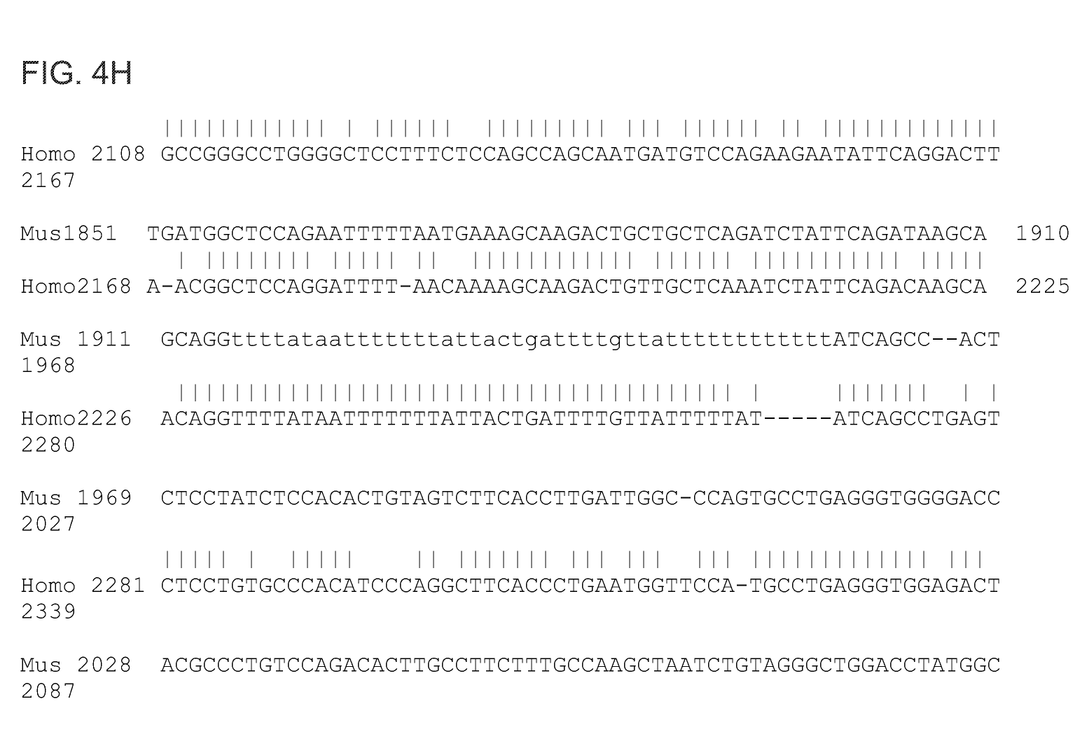

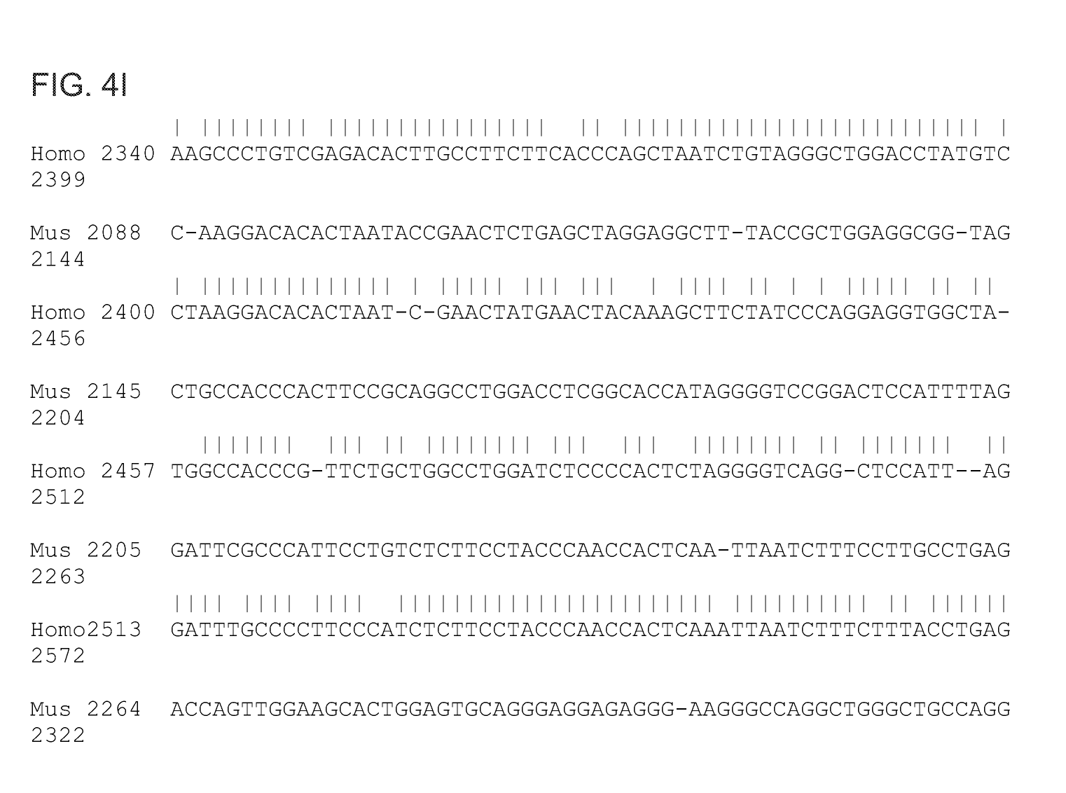

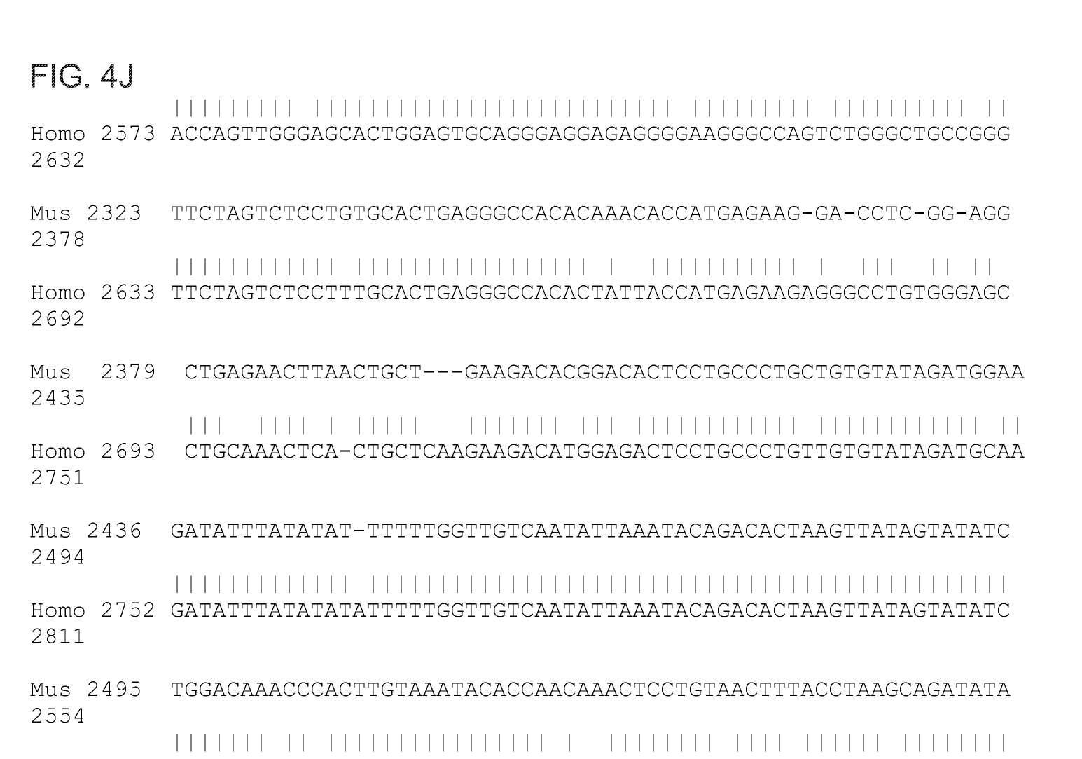

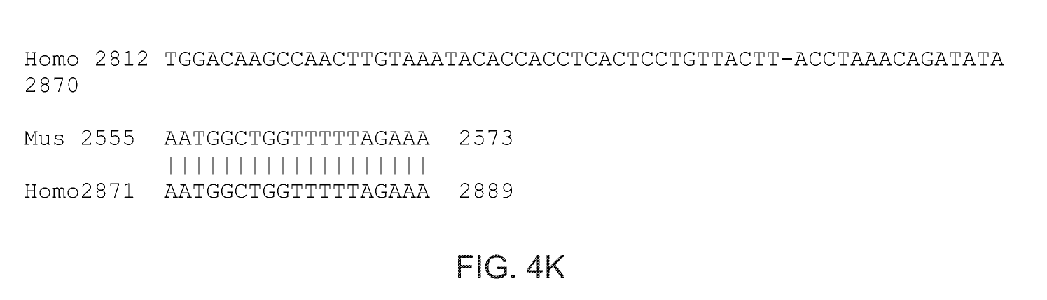

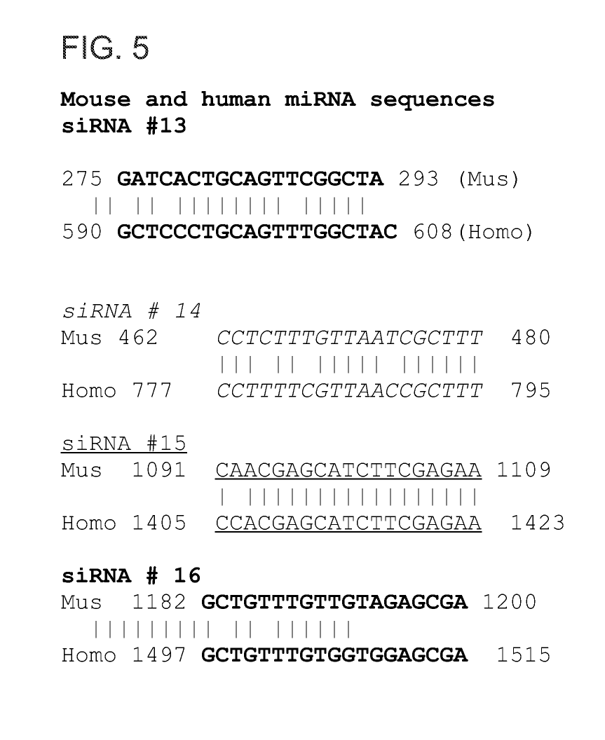

GLUT1 is present on many cells including brain vascular endothelium (Pardridge et al., 1990), but it is unclear whether it is essential for glucose transport into brain as it is for retina. To test this, additional studies were performed in which the effect of GLUT1 blockade in diabetics on glucose levels in brain as well as retina and RBCs was measured. Compared to nondiabetics, mice diabetic for 2 months had a significant increase in retinal and RBC glucose levels (FIG. 3A), but brain glucose levels were not significantly different in diabetics compared to nondiabetics (FIG. 3B). Mice diabetic for 2 months that were treated with forskolin or 1,9-dideoxyforskolin had significant reduction in glucose levels in retina and RBCs, but not in brain (FIGS. 3A and 3B). The rank order of glucose level normalized to tissue protein level was brain>retina>RBC, because the rank order of protein content per wet weight was RBC>retina>brain. This is because lipid content is greatest in brain, intermediate in retina, and lowest in RBC.

Since the genistein blocks GLUT1 transport by binding to the ATP binding site which is distinct from the forskolin binding site (Vera et al., 2001), combined use of these agents could potentially provide greater blockade of GLUT1 than forskolin alone. Another experiment was performed to determine if this combination treatment could reduce glucose levels in brain, and to try to further maximize GLUT1 also added myricetin, another isoflavone that binds to the ATP binding site of GLUT1 (Vera et al., 2001). In this experiment, reversibility of GLUT1 blockade by varying the time between last dose and euthanasia was tested. Mice treated with forskolin, genistein, and myricetin during 2 months of diabetes showed a significant reduction in glucose levels in retina and RBCs 5 hr after the last dose compared to untreated diabetics (FIG. 3C). There was still a significant reduction in retinal and RBC glucose levels at 24 and 48 hr after the last dose, but levels were significantly higher at 48 hr compared to 5 hr. Similar to the previous experiment, there was no significant increase in brain glucose levels in diabetics compared to nondiabetics (FIG. 3D). Combined treatment with forskolin, genistein, and myricetin had no effect on brain glucose levels and the levels were the same 5, 24, and 48 hr after the last dose.

The results reported herein were obtained using the following methods and materials.

### Mice

In male C57BL/6 mice (Harlan Laboratories, Indianapolis, Ind.) at 5 weeks of age, diabetes was induced by intraperitoneal (IP) injection on four consecutive days of 50 mg/kg of freshly prepared streptozotocin (STZ; Calbiochem, San Diego, Calif.) in sodium citrate buffer (pH 4.5). Development of diabetes (defined as blood glucose >250 mg/dl) was verified 1 week after the final STZ injection by measurement of blood glucose level with an Accu-Chek Aviva glucometer (Roche Diagnostics, Indianapolis, Ind.). Mice were housed in a pathogen-free facility with a 12-hr light/dark cycle and free access to food and water.

### Knock Down of GLUT1

Mice that had been diabetic for several weeks were given a 1 μl intravitreous injection of vehicle in one eye and 1 μg of a mixture of three siRNAs directed against SLC2A1 mRNA which codes for GLUT1 (On-Target plus SMART siRNA pool, Thermo Scientific, Lafayette, Colo.) in the other eye on days 1, 4, and 7. The mice were euthanized on day 10, retinas were dissected and GLUT1 was measured by immunoblot. Retinal glucose levels were measured as described below.

### Inhibition of GLUT1

Diabetic mice were given daily IP injections of 0.1, 0.5, 1, or 5 mg/kg of freshly prepared forskolin, 0.1 mg/kg of 1,9-dideoxyforskolin, 50 mg/kg of genistein, 1 mg/kg myricetin in PBS/0.1% DMSO (all from Sigma, Saint Louis, Mo.) or a combination of these and after various time periods, retinas, red blood cells (RBCs), and white blood cells (WBCs) were collected for assays described below.

### Glucose Measurements in Retina, Brain, and Red Blood Cells (RBC)

Glucose in retinal and RBC homogenates was measured by Glucose Assay kit (Sigma). Briefly, mice were euthanized and blood was removed from the heart into a heparinized tube and centrifuged at 600 g for 5 min at 4° C. The buffy coat was collected and stored for assays in WBCs. The RBCs were washed three times with isotonic buffer (0.9% NaCl/PBS). Retinas or brains were dissected and washed and two retinas were placed in 210 μl of H₂O. After three freeze/thaw cycles and homogenization, samples were centrifuged at 14,000 rpm for 10 min at 4° C. and the glucose concentration of the supernatant was measured using the manufacturer's instructions. Protein was measured with the BIO-RAD kit (BIO-RAD Life Science Research, Hercules, Calif.) using the manufacturer's instructions.

### Measurement of the Glycohemoglobin

Red blood cells (RBCs) were sonicated and centrifuged and the supernatant containing hemoglobin was collected. Protein concentration was measured with a protein assay kit (BioRad, Hercules, Calif.) and 1 mg of protein was applied to a ConA column of a glycoprotein isolation kit (Thermo Scientific, Rockford, Ill.). The columns were washed and glycoproteins were eluted and measured using the manufacturer's instructions.

### Immunoblots

Retinas were dissected and two retinas were placed in 50 μl of lysis buffer (10 mmol/L Tris, pH 7.2, 0.5% Triton X-100, 50 mmol/L NaCl, and 1 mmol/L EDTA containing a proteinase inhibitor mixture tablet (Roche, Indianapolis, Ind.). After three freeze/thaw cycles and homogenization, samples were centrifuged at 14,000 rpm for 10 min at 4° C. and the protein concentration of the supernatant was measured. For each sample, 50 μg of protein was resolved by sodium dodecyl sulfate-polyacrylamide gel electrophoresis (BioRad), and transferred to a nitrocellulose membrane (Hybond-ECL; Amersham Biosciences, Piscataway, N.J.). Rabbit polyclonal anti-mouse GLUT1 (1:1,000; Chemicon International, Temecula, Calif.), was used as primary antibody. The secondary antibody was a horseradish peroxidase-coupled goat anti-rabbit IgG (1:2,000; Cell Signaling, Danvers, Mass.). Blots were incubated in SuperSignal Western Pico Lumino/Enhancer solution (Pierce, Rockford, Ill.) and exposed to X-ray film (Eastman-Kodak, Rochester, N.Y.). To assess loading levels of protein, GLUT1 blots were stripped and incubated with polyclonal rabbit anti-β-actin antibody (1:5,000; Cell Signaling) followed by horseradish peroxidase-coupled goat anti-rabbit IgG.

### Real-Time RT-PCR

In some experiments, one eye was used for measurement of glucose level, and the fellow eye was used to measure levels of mRNA by real-time RT-PCR (Shen et al., 2006). Cyclophilin was amplified for normalization. The following primers were used: (1) CRYBB2: forward, 5′-TCT GAG GCC CAT CAA AGT GGA CAG CC-3′ and reverse, 5′-ACG CAC GGA AGA CAC CTT TTC CTG GTA-3′, (2) VEGF: forward, 5′-CAC GAC AGA AGG AGA GCA GAAG-3′ and reverse, 5′-ACA CAG GAC GGC TTG AAG ATG-3′, (3) cyclophilin, forward, 5′-CAG ACG CCA CTG TCG CTT T-3′ and reverse, 5′-TGT CTT TGG AAC TTT GTC TGC AA-3′.

### Superoxide Radical Assay

Superoxide radicals were measured in retinal homogenates by measuring dihydroethidine-induced fluorescence (Georgiou et al., 2008; Chen et al., 2009). Briefly, 200 μl reaction mixtures of freshly prepared retinal homogenates (20 μg protein) containing 2.5 μM dihydroethidium and 0.05 μg/μl salmon testes DNA were placed in 96-well microplates at 37° C. for 30 min. The fluorescence was measured at an excitation of 480 nm and an emission of 610 nm. Each sample was assayed in triplicate.

### OTHER EMBODIMENTS

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

1. A method of treating or preventing a diabetic complication in a subject, the method comprising administering to a subject in need thereof an effective amount of a Glut1 antagonist selected from the group consisting of inhibitory nucleic acids, antibodies that specifically bind Glut1, and small compounds.

2. The method of claim 1, wherein the diabetic complication is retinopathy, nephropathy, neuropathy, or acceleration of atherosclerosis.

3. A method of treating or preventing diabetic retinopathy in a subject, the method comprising administering to a subject in need thereof an effective amount of a Glut1 antagonist selected from the group consisting of inhibitory nucleic acids, antibodies that specifically bind GLUT1, and small compounds.

4. The method of claim 1, wherein the sequence of the inhibitory nucleic acid is:
Homo sapiens siRNA 777 CCTTTTCGTTAACCGCTTT 795.

5. The method of claim 1, wherein the small compound is selected from the group consisting of myricetin, genistein, 1,9-dideoxyforskolin, and forskolin.

6. The method of claim 1, wherein the inhibitory nucleic acid is a siRNA, shRNA, or antisense polynucleotide.

7. The method of claim 6, wherein the inhibitory nucleic acid molecule is expressed in a vector.

8. The method of claim, 1 wherein the subject is diagnosed as having or having a propensity to develop type I or type II diabetes, hyperglycemia, or a pre-diabetic condition.

9. The method of claim 1 wherein the Glut1 antagonist is administered orally or intra-ocularly.

10. The method of claim 1 wherein the Glut1 antagonist is administered systemically.

11. The method of claim 1 wherein the administration prevents elevation of superoxide radicals, increased expression of the chaperone protein β2 crystallin, and/or increased expression of vascular endothelial growth factor (VEGF).

12. An expression vector encoding a glut1 inhibitory nucleic acid molecule.

13. The expression vector of claim 12, wherein the vector encodes an inhibitory nucleic acid is a siRNA, shRNA, or antisense polynucleotide.

14. The expression vector of claim 12, wherein the vector is a adenoviral 2 or 9 vector.

15. A method for monitoring Glut1 levels in a subject receiving a Glut1 antagonist, the method comprising measuring glucose and/or glycohemoglobin levels in a red blood cells of the subject and comparing said levels to a reference.

16. The method of claim 15, wherein the reference is the level of glucose and/or glycohemoglobin present in a healthy control subject.

17. The method of claim 15, wherein the reference is the level of glucose and/or glycohemoglobin present in the subject prior to treatment with a Glut1 antagonist.

18. The method of claim 15, wherein the reference is the level of glucose and/or glycohemoglobin present in the subject at an earlier time point.

19. The method of claim 15, wherein a reduction in glucose and/or glycohemoglobin levels relative to the reference indicates that Glut1 antagonist therapy is efficacious.

20. An ocular formulation comprising a Glut1 antagonist selected from the group consisting of inhibitory nucleic acids, antibodies that specifically bind Glut1, and small compounds.

21. The ocular formulation of claim 16, wherein the Glut1 antagonist is selected from the group consisting of inhibitory nucleic acids, antibodies that specifically bind Glut1, and small compounds.

22. The ocular formulation of claim 16, wherein the small compound is selected from the group consisting of genistein, 1,9-dideoxyforskolin, and forskolin.

23. The ocular formulation of claim 16, wherein the inhibitory nucleic acid is a siRNA, shRNA, or antisense polynucleotide.
